SEQUENCE LISTING (1 sequence)

SEQ ID NO: 1 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

(19) United States
(12) Patent Application Publication
(10) Pub. No.: US 2013/0028978 A1
(43) Pub. Date: Jan. 31, 2013

(54) COMPOSITIONS AND METHODS FOR WOUND TREATMENT

(71) Applicants: Jeremy J. Mao, Closter, NJ (US); Chang Hun Lee, New York, NY (US)

(72) Inventors: Jeremy J. Mao, Closter, NJ (US); Chang Hun Lee, New York, NY (US)

(73) Assignee: The Trustees of Columbia University in the City of New York, New York, NY (US)

(21) Appl. No.: 13/509,231

(22) PCT Filed: Nov. 10, 2010
(86) PCT No.: PCT/US10/56250

§ 371 Date: Oct. 17, 2012

Related U.S. Application Data
(60) Provisional application No. 61/259,822, filed on Nov. 10, 2009.

(51) Int. Cl. A61K 38/18; A61K 35/12; A61K 39/395; C12N 5/0775; A61K 48/00; A61K 38/16; A61K 9/14; A61P 17/02; A61K 31/711; A61K 31/713

(52) U.S. Cl. 424/497; 514/9.4; 514/44 R; 424/93.7; 424/142.1; 424/158.1; 514/44 A; 514/2.3; 435/377

(57) ABSTRACT

Provided herein are compositions, methods, systems, and kits for wound healing. As shown herein, CCN2/CTGF stimulated mesenchymal progenitor cells can form αSMA⁻ fibroblasts. Further, TGFβ was shown to stimulate further differentiation of αSMA⁻ fibroblasts to myofibroblasts associated with fibrosis. One aspect provides a composition including CCN2/CTGF and a TGFβ inhibitor, a P38 inhibitor, or a tyrosine kinase inhibitor. Another aspect provides a method of treating tissue wounds with CCN2/CTGF-containing compositions. Also provided are systems and kits for wound healing. Also provided are methods for forming αSMA⁻ fibroblasts mesenchymal progenitor cells.

### CROSS-REFERENCE TO RELATED APPLICATIONS

The present application claims the benefit of U.S. Provisional Application Ser. No. 61/259,822 filed 10 Nov. 2009; which is incorporated herein by reference in its entirety.

### STATEMENT REGARDING FEDERALLY SPONSORED RESEARCH OR DEVELOPMENT

This invention was made with Government support under R01DE15391 awarded by the National Institutes of Health. The Government has certain rights in the invention.

### INCORPORATION-BY-REFERENCE OF MATERIAL SUBMITTED IN COMPUTER READABLE FORM

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present invention generally relates to tissue wound treatment.

### BACKGROUND

Fibroblasts are ubiquitous cells and constitute the stroma of virtually all tissues. Due to their broad distribution from hair to toe, fibroblasts in different tissues express heterogeneous genotypes (1). In addition to collagen producing cells, fibroblasts are reported to interact with immune cells, inflammatory cells. Cancer associated fibroblasts have a putative role in cancer stroma (2-4). Cancer associated fibroblasts may immobilize invasive tumor cells and elaborate vasculogenesis, both of which are prerequisite for metastasis (3, 4). Organ fibrosis is typically characterized by the transformation of epithelial or endothelial cells into fibroblasts which further acquire a myofibroblast phenotype (5-7).

Fibroblast contraction of granular tissue is a process of normal wound healing (6, 8). In pathological wound healing, the activation of fibroblasts by acquiring α-smooth muscle actin (αSMA) phenotype and excessive contractility are among the factors responsible for fibrosis or aberrant scarring (9-11), including keloids and hypertrophic scars to which there is currently no satisfactory therapy (10, 12).

Besides stromal cells, fibroblasts also act as parenthymal cells in several specialized connective tissues such as ligaments, tendons and the periodontal ligament. In contrast to excessive collagen biosynthesis in organ fibrosis in which fibroblasts are stromal cells, parenthymal fibroblastic tissues such as tendons and ligaments are recalcitrant to regeneration (13-15). The poor innate healing capacity of parenthymal fibroblastic tissues is attributed to the scarcity of fibroblasts as collagen producing cells (13-15). Recently skin fibroblasts were transformed into induced pluripotent stem cells (iPS) (16, 17). However the yield of reprogramming is generally low, leading to the speculation that postnatal stem/progenitor cells among the fibroblast population, rather than end-stage fibroblasts, are readily reprogrammed. Despite their widespread use in cell biology and broad implications in diseases such as cancer and fibrosis, fibroblasts are not well studied cells regarding their origin(s) and differentiation pathways.

It has been proposed that fibroblasts may derive from epithelial or endothelial cells in a process dubbed as endothelial- or epithelial-mesenchymal transition (EMT) (18-22). Cell tracing in transgenic models showed the transformation of endothelial or epithelial cells into fibroblast-like cells (23). But EMT does not account for all the fibroblasts that are present in organ fibrosis (24). Also, EMT does not explain the origin of parenthymal fibroblasts, given the paucity of either epithelial or endothelial cells in tendons or ligaments. Recently, multipotent mesenchymal cells were discovered in tendons (25), supporting the hypothesis of the mesenchymal origin of fibroblasts (26-28).

A putative mesenchymal origin of fibroblasts can be either bone marrow derived or connective tissue derived. In bone marrow, CD34+ and CD45+ fibrocytes are regarded as a subpopulation of stem/progenitor cells with characteristics of hematopoietic stem cells, monocytes and fibroblasts, and may migrate to the periphery upon wounding (6, 29). However, CD34+ and CD45+ fibrocytes only account for <1% of total bone marrow cells (6), and are likely not involved in the homeostasis of connective tissues throughout the body. Thus, the origin and differentiation pathways of fibroblasts responsible for the homeostasis and repair of connective tissues upon insults such as trauma, cancer and infection remain enigmatic.

To date, there is no reliable and convenient way to differentiate stem cells or progenitor cells into fibroblasts. And, to date, it has not been reported that fibroblasts can derive from epithelial cells or endothelial cells in organ fibrosis.

### SUMMARY OF THE INVENTION

Among the various aspects of the present invention is the provision of compositions and methods for wound treatment.

One aspect provides a pharmaceutical composition including CCN2/CTGF and a pharmaceutically acceptable carrier or excipient. In some embodiments, the pharmaceutical composition includes CCN2/CTGF. In some embodiments, the pharmaceutical composition includes CCN2/CTGF and an inhibitor of TGFβ. In some embodiments, the pharmaceutical composition includes CCN2/CTGF and a P38 inhibitor. In some embodiments, the pharmaceutical composition includes CCN2/CTGF and tyrosine kinase inhibitor. In some embodiments, the pharmaceutical composition includes CCN2/CTGF and two or more of an inhibitor of TGFβ, a P38 inhibitor, or a tyrosine kinase inhibitor.

In some embodiments, the pharmaceutical composition includes a mesenchymal progenitor cell. In various embodiments, the mesenchymal progenitor cell is a αSMA− mesenchymal progenitor cell. In various embodiments, the mesenchymal progenitor cell is a CD34− mesenchymal progenitor cell.

In some embodiments, the CCN2/CTGF comprises a CCN2/CTGF polypeptide or a polynucleotide encoding a CCN2/CTGF polypeptide. In some embodiments, the composition includes a polynucleotide encoding a CCN2/CTGF polypeptide operably linked to a vector. In some configurations, the vector is suitable for expression of the CCN2/CTGF polypeptide in a wound tissue environment.

In some embodiments, the CCN2/CTGF includes human CCN2/CTGF or recombinant human CCN2/CTGF. In some embodiments, the CCN2/CTGF includes a CCN2/CTGF corresponding to Accession No. NP_{—}001892. In some embodiments, the CCN2/CTGF includes a polypeptide having a sequence of SEQ ID NO: 1, or at least about 80% identity thereto and CCN2/CTGF activity. In some embodiments, the CCN2/CTGF includes a polypeptide having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1 and CCN2/CTGF activity.

In some embodiments, the composition includes an inhibitor of TGFβ. In some embodiments, the inhibitor of TGFβ reduces formation of myofibroblasts from fibroblasts or inhibits fibrosis. In some embodiments, the inhibitor of TGFβ substantially reduces formation of myofibroblasts from fibroblasts or inhibits fibrosis. In some embodiments, the composition includes an inhibitor of TGFβ1. In some embodiments, the composition includes an inhibitor of TGFβ2. In some embodiments, the composition includes an inhibitor of TGFβ3. In some embodiments, the composition includes an inhibitor of TGFβ3 selected from the group consisting of ANG-1122; AP-11014; metelimumab; fresolimumab; mannose-6-phosphate, BTG; Pharmaprojects No. 6614; NAFB001; NAFB002; TGF-β1 antibody, Lilly; LY-2157299; Fetuin; TGF-β antagonists, FibroGen; 1D11; anti-TGFβ MAb-1, Genzyme; SB-431542; activin-like kinase 5 inhibitor, Graceway; anti-TGF-β antibodies, Genent; antisense oligonucleotide, II; TGF-β antagonists, Inflazyme; TGF-β receptor, Insmed; TGF-β antagonists, Inspiraplex; decorin, Telios; SX-007; TGF-β receptor inhibs, J&J; TGF-β vaccine, Neovacs; ADMP-1; TGF-β antibodies, Manchester; TGF-β antagonists, Sydney; mannose-6-phosphonate, Renovo; cancer gene therapy, Resver; TGF-Beta Shield; IN-1130; LF-984; TGF-β inhibitors, Mill; and SB-431542.

In some embodiments, the composition includes a P38 inhibitor. In some embodiments, the composition includes a P38 inhibitor selected from the group consisting of Tocriset, SD282, SB239063, SB203580, SB220025, SKF86002, PD169316, SB202190, SC68376, VX702, VX745, R130823, AMG548, BIRB796, SCIO469, SCIO323, FR167653, MW12069ASRM, SD169, RWJ67657, and ARRY797.

In some embodiments, the composition includes a tyrosine kinase inhibitor. In some embodiments, the composition includes a tyrosine kinase inhibitor selected from the group consisting of K252a, Axitinib, Bosutinib, Cediranib, Dasatinib, Erlotinib, Gefitinib, Imatinib, Lapatinib, Lestaurtinib, Nilotinib, Semaxanib, Sunitinib, Toceranib, Vandetanib, Vatalanib, ZD 1839, CI-1033, OSI-774, GW 2016, EKB-569, IMC-C225, MDX-447, PKI 116, ABX-EGF, AG-82, AG-18, AG-490, AG-17, AG-213, AG-494, AG-825, AG-879, AG-1112, AG-1296, AG-1478, AG-126, RG-13022, RG-14620, and AG-555.

In some embodiments, the composition includes an antibiotic or an immunosuppressive agent. In some embodiments, the composition includes an antibiotic selected from the group consisting of amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin. In some embodiments, the composition includes an immunosuppressive agent selected from the group consisting of a steroid, cyclosporine, cyclosporine analog, cyclophosphamide, methylprednisone, prednisone, azathioprine, FK-506, 15-deoxyspergualin, prednisolone, methotrexate, thalidomide, methoxsalen, rapamycin, leflunomide, mizoribine, brequinar, deoxyspergualin, azaspirane, muromonab-CD3, Sandimmune, Neoral, Sangdya, Prograf, Cellcept, azathioprine, glucocorticosteroids, adrenocortical steroid, Deltasone, Hydeltrasol, Folex, methotrexate, methoxsalen, and sirolimus.

In some embodiments, the composition is formulated for parenteral, pulmonary, oral, topical, intradermal, intramuscular, intraperitoneal, intravenous, subcutaneous, intranasal, epidural, ophthalmic, buccal, or rectal administration. In some embodiments, the composition is formulated for topical administration. In some embodiments, the composition is formulated for topical administration directly to a soft tissue wound site.

One aspect provides a a kit for wound treatment. In some embodiments, the kit includes one or more compositions described above. In some embodiments, the kit includes one or more pharmaceutical compositions described above.

One aspect provides the use of a composition described above for treatment of a tissue wound. In some embodiments, a pharmaceutical composition described above is used for treatment of a tissue wound.

One aspect provides the use of a composition described above in the manufacture of a medicament for the treatment of a tissue wound. In some embodiments, a pharmaceutical composition described above is used in the manufacture of a medicament for the treatment of a tissue wound.

In some embodiments, the tissue wound includes a soft tissue wound. In some embodiments, the tissue wound includes one or more of a chronic tissue wound or an acute tissue wound. In some embodiments, the tissue wound includes one or more of a dermal wound, a ligament wound, a tendon wound, or a combination thereof. In some embodiments, the tissue wound includes an open tissue wound. In some embodiments, the tissue wound includes one or more of an incision wound, a laceration wound, an abrasion wound, a puncture wound, a penetration wound, or a gunshot wound. In some embodiments, the tissue wound includes one or more of a split laceration, over stretching, grinding compression, cut laceration, or tearing.

One aspect provides a system for healing a wound at a tissue site. In some embodiments, the system includes a medical device and a composition described above. In some embodiments, the system includes a medical device and a pharmaceutical composition described above. In some embodiments, the composition is adapted to be released from the medical device when in contact with a tissue site. In some embodiments, the medical device includes one or more of a drape, bandage, dressing, tape, adhesive layer, splint, blood stop powder, steri strip, cyanoacrylate glue, staple, and suture, or a combination thereof.

One aspect provides a method of treating a subject. In some embodiments, the method includes administering a composition including CCN2/CTGF to a tissue wound site in a subject in need thereof. In some embodiments, the method includes administering a composition comprising αSMA− fibroblast (derived from CCN2/CTGF treated mesenchymal progenitor cells) to a tissue wound site in a subject in need thereof. In some embodiments, the method includes contacting the composition including CCN2/CTGF and a mesenchymal progenitor cell to stimulate fibroblast differentiation. In some embodiments, the method includes administering a composition that includes a mesenchymal progenitor cell. In some embodiments, the mesenchymal progenitor cell is a αSMA− mesenchymal progenitor cell. In some embodiments, the mesenchymal progenitor cell is a CD34− mesenchymal progenitor cell. In some embodiments, the mesenchymal progenitor cell is autogeneic, allogeneic, isogeneic, or xengeneic, or a combination thereof.

In some embodiments, differentiated fibroblasts are included in compositions or administered to a subject. In some embodiments, differentiated fibroblasts are αSMA− fibroblasts. In some embodiments, the differentiated fibroblasts are FSP1+, vimentin+, Coll1+ and αSMA−. In some embodiments, the differentiated fibroblasts are present in an enriched cell culture that includes at least about 80%, 85%, 90%, 95%, or 99% differentiated fibroblasts.

In some embodiments, the method of treating a subject includes administering at least one or more of (i) an inhibitor of TGFβ; (ii) a P38 inhibitor; or (iii) a tyrosine kinase inhibitor. In some embodiments, the method of treating a subject includes administering an inhibitor of TGFβ. In some embodiments, the method of treating a subject includes administering a P38 inhibitor. In some embodiments, the method of treating a subject includes administering a tyrosine kinase inhibitor.

In some embodiments, the method includes administering a composition that includes CCN2/CTGF and at least one of the inhibitor of TGFβ, the P38 inhibitor, or the tyrosine kinase inhibitor. In some embodiments, a first composition includes CCN2/CTGF and a second composition includes at least one of the inhibitor of TGFβ, the P38 inhibitor, or the tyrosine kinase inhibitor. In some embodiments, the first composition and the second composition are administered consecutively. In some embodiments, the first composition and the second composition are administered simultaneously.
In some embodiments, at least one of the inhibitor of TGFβ, the P38 inhibitor, or the tyrosine kinase inhibitor inhibits fibrosis. In some embodiments, the inhibitor of TGFβ inhibits fibrosis. In some embodiments, the P38 inhibitor inhibits fibrosis. In some embodiments, the tyrosine kinase inhibitor inhibits fibrosis.
In some embodiments, the method includes administering a composition that includes an inhibitor of TGFβ. In some embodiments, the inhibitor of TGFβ reduces formation of myofibroblasts from fibroblasts or inhibits fibrosis. In some embodiments, the inhibitor of TGFβ is present in an amount effective to substantially reduce formation of myofibroblasts from fibroblasts. In some embodiments, the inhibitor is an inhibitor of TGFβ. In some embodiments, the inhibitor is an inhibitor of TGFβ2. In some embodiments, the inhibitor is an inhibitor of TGFβ3. In some embodiments, the inhibitor of TGFβ is selected from the group consisting of ANG-1122; AP-11014; metelimumab; fresolimumab; mannose-6-phosphate, BTG; Pharmaprojects No. 6614; NAFB001; NAFB002; TGF-β1 antibody, Lilly; LY-2157299; Fetuin; TGF-β antagonists, FibroGen; 1D11; anti-TGFβ MAb-1, Genzyme; SB-431542; activin-like kinase 5 inhibitor, Graceway; anti-TGF-β antibodies, Genent; antisense oligonucleotide, II; TGF-β antagonists, Inflazyme; TGF-β receptor, Insmed; TGF-β antagonists, Inspiraplex; decorin, Telios; SX-007; TGF-β receptor inhibs, J&J; TGF-β vaccine, Neovacs; ADMP-1; TGF-β antibodies, Manchester; TGF-β antagonists, Sydney; mannose-6-phosphonate, Renovo; cancer gene therapy, Resver; TGF-Beta Shield; IN-1130; LF-984; TGF-β inhibitors, Mill; and SB-431542.
In some embodiments, the method includes administering a composition that includes a P38 inhibitor. In some embodiments, the P38 inhibitor is one or more of Tocriset, SD282, SB239063, SB203580, SB220025, SKF86002, PD169316, SB202190, SC68376, VX702, VX745, R130823, AMG548, BIRB796, SCIO469, SCIO323, FR167653, MWO12069ASRM, SD169, RWJ67657, and ARRY797.
In some embodiments, the method includes administering a composition that includes a tyrosine kinase inhibitor. In some embodiments, tyrosine kinase inhibitor is one or more of K252a, Axitinib, Bosutinib, Cediranib, Dasatinib, Erlotinib, Gefitinib, Imatinib, Lapatinib, Lestaurtinib, Nilotinib, Semaxanib, Sunitinib, Toceranib, Vandetanib, Vatalanib, ZD 1839, CI-1033, OSI-774, GW 2016, EKB-569, IMC-C225, MDX-447, PKI 116, ABX-EGF, AG-82, AG-18, AG-490, AG-17, AG-213, AG-494, AG-825, AG-879, AG-1112, AG-1296, AG-1478, AG-126, RG-13022, RG-14620, and AG-555.
In some embodiments, the method treats a tissue wound site that includes a soft tissue wound. In some embodiments, the tissue wound site includes a chronic soft tissue wound or an acute soft tissue wound. In some embodiments, the tissue wound site includes one or more of a dermal wound, a ligament wound, a tendon wound, or a combination thereof. In some embodiments, the tissue wound site includes an open tissue wound. In some embodiments, the tissue wound site includes one or more of an incision wound, a laceration wound, an abrasion wound, a puncture wound, a penetration wound, or a gunshot wound. In some embodiments, the tissue wound site includes one or more of a split laceration, over stretching, grinding compression, cut laceration, or tearing.
In some embodiments, the method includes parenteral, pulmonary, oral, topical, intradermal, intramuscular, intraperitoneal, intravenous, subcutaneous, intranasal, epidural, ophthalmic, buccal, or rectal administration. In some embodiments, administration includes topical administration. In some embodiments, administration includes topical administration directly to a tissue wound site.
In some embodiments, administering the composition results in enhancement of fibroblast differentiation. In some embodiments, administering the composition results in enhancement of fibrogenesis. In some embodiments, administering the composition results in inhibition of myofibroblast differentiation. In some embodiments, administering the composition results in inhibition of fibrosis. In some embodiments, administering the composition results in at least one of enhancement of fibroblast differentiation; enhancement of fibrogenesis; inhibition of myofibroblast differentiation; or inhibition of fibrosis. In some embodiments, administering the composition results in enhancement of fibroblast differentiation; enhancement of fibrogenesis; inhibition of myofibroblast differentiation; and inhibition of fibrosis.
In some embodiments, the method includes administering any of the compositions described above. In some embodiments, administering the composition comprises administering any of the systems described above to a tissue wound site.
In some embodiments, the composition is administered via a carrier delivery system. In some embodiments, the composition is administered via a carrier delivery system. In some embodiments, the composition is administered via a carrier delivery system including polymeric microspheres, and the composition is encapsulated in the polymeric microspheres. In some embodiments, the composition is encapsulated in polymeric microspheres at a ratio of about 100 mg to about 500 mg polymer to about 1 to about 100 μg of CCN2/CTGF. In some embodiments, the composition is encapsulated in polymeric microspheres at a ratio of about 250 mg polymer to about 10 μg of CCN2/CTGF. In some embodiments, administering the composition comprises introducing about 1 to about 50 mg of CTGF-encapsulated microspheres to a a tissue wound.
One aspect provides a method of forming an αSMA− fibroblast. In some embodiments, the method includes contacting a αSMA−, CD34− mesenchymal progenitor cell and CCN2/CTGF, wherein the CCN2/CTGF-stimulated mesenchymal stem cell differentiates into a αSMA−, FSP1+, vimentin+, Coll1+ fibroblast cell.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a series of images and bar graphs showing CCN2/CTGF mediated fibroblastic differentiation of mesenchymal cells. FIG. 1A is an image of isolated and culture-expanded bone marrow mesenchymal stem cells (MSCs). Scale: 100 μm. FIG. 1B is an image of trichrome stained isolated and culture-expanded bone marrow mesenchymal stem cells treated with CCN2/CTGF (100 ng/mL), which prompted substantial collagen synthesis (compare with FIG. 1A showing MSCs without CCN2/CTGF treatment). Scale: 100 μm. FIG. 1C is a bar graph showing collagen type I and FIG. 1D is a bar graph showing tenascin-C contents of CCN2/CTGF-treated MSCs cells, which were significantly higher than MSCs without CCN2/CTGF treatment at the tested 2 and 4 wks (n=5, *:p<0.05, **:p<0.01). FIG. 1E is a series of images showing collagen deposition increased with increasing CCN2/CTGF doses from 0-100 ng/mL. FIG. 1F is a bar graph showing MSC surface markers (including CD29, CD44, CD105, CD106, CD117, BMPR and Sca1) gradually attenuated upon 2 and 4 wks of CCN2/CTGF treatment (p<0.05). FIG. 1G is a bar graph showing that in parallel, fibroblastic markers gradually increased including collagen types I and III, Tn-C, fibronectin, matrix metalloproteinase 1 (MMP-1), fibroblast specific protein 1 (FSP1) and vimentin upon 2 and 4 wks of CCN2/CTGF treatment. In FIG. 1G, a chondrogenic marker (collagen type II) and myofibroblastic marker (αSMA) were undetectable, whereas an osteogenic marker (osteopontin) was minimally expressed.

FIG. 2 is a series of images showing clonal differentiation of CCN2/CTGF-treated MSCs and attenuated differentiation ability of CCN2/CTGF-treated MSCs into other mesenchymal lineages. CCN2/CTGF-treated MSCs (MSC-Fb) (for 4 wks) showed minimal capacity to further differentiate into osteoblasts stained with Alizarin Red (FIG. 2A), chondrocytes stained with Safarinin-O (FIG. 2B) or adipocytes stained with Oil-Red O (FIG. 2C). In contrast, the same subpopulation of MSCs without CCN2/CTGF treatment was readily differentiated into osteoblasts (FIG. 2D), chondrocytes (FIG. 2E) and adipocytes (FIG. 2F). To address the heterogeneity of MSCs, single clones were established (B7, B12 and E3), and differentiated into fibroblastic (FIG. 2G, FIG. 2H, FIG. 2I), osteogenic (FIG. 2J, FIG. 2K, FIG. 2L), adipogenic (FIG. 2M, FIG. 2N, FIG. 2O), chondrogenic cells (FIG. 2, FIG. 2Q, FIG. 2R). Scale: 50 μm (FIG. 2C, FIG. 2F, FIGS. 2M-O); 100 μm (all others).

FIG. 3 is a series of images and line and scatter plots showing myofibroblastic differentiation of MSC-derived fibroblastic cells by TGFβ1. Either undifferentiated MSCs or CCN2/CTGF-treated MSCs expressed minimal alpha-smooth muscle actin (αSMA) (FIG. 3A, FIG. 3C). Upon TGFβ1 treatment, MSCs or MSC-derived fibroblasts readily expressed αSMA+ microfilaments (FIG. 3B, FIG. 3D). Flow cytometry confirmed the absence of αSMA expression in MSCs or MSC-derived fibroblasts (MSC-Fb) (FIG. 3E, FIG. 3G). Contrastingly, 31.9% of CCN2/CTGF-treated MSCs or MSC-Fb gained αSMA phenotype (FIG. 3H). Strikingly, only 1.8% of undifferentiated MSCs gained αSMA phenotype (FIG. 3F) after TGFβ1 stimulation. Collagen gel contraction assay showed that MSCs with sequential administration of CCN2/CTGF and TGFβ1 yielded the most significant contraction (FIG. 3I), in comparison with moderate contraction upon CCN2/CTGF stimulation alone (FIG. 3J), or TGFβ1 stimulation alone of undifferentiated MSCs (FIG. 3K). MSCs without either CCN2/CTGF or TGFβ1 stimulation yielded the least contraction (FIG. 3L). Quantitatively, sequential stimulation of MSCs by CCN2/CTGF and TGFβ1 yielded the most significant contraction of collagen gel (FIG. 3M) (p<0.05). Scale: 100 μm.

FIG. 4 is a series of images and a line and scatter plot showing CCN2/CTGF promotes fibrogenesis instead of ectopic osteogenesis in vivo. In FIG. 4A, ectopic mineralization readily occurs (box) following resection of a synostosed calvarial suture on the representative 3D reconstructed pCT image. In FIG. 4B, the anatomic morphology of a calvarial suture was restored with absence of ectopic mineralization upon controlled release of CCN2/CTGF. CCN2/CTGF-encapsulated PLGA microspheres were 120±64 μm in diameter per SEM image (FIG. 4C), and showed sustained release up to the tested 6 wks in vitro (n=6) (FIG. 4D). H&E staining showed that microscopic morphology of the calvarial suture was restored upon controlled release of CCN2/CTGF (FIG. 4F, FIG. 4H), in comparison with ectopic mineralization without CCN2/CTGF delivery (FIG. 4E, FIG. 4G). Some of CCN2/CTGF-encapsulated microspheres (μs) remained present 4 wks post-op (FIG. 4F, FIG. 4H). Abundant FSP1 and vimentin expression (FIG. 4J, FIG. 4L, respectively) indicates the presence of fibroblast-like cells in regenerated calvarial suture. Contrastingly, FSP1 and vimentin expression was restricted to the marrow of obliterated bone (FIG. 4I, FIG. 4K) without CCN2/CTGF delivery. Scale: 1 mm (FIG. 4A, FIG. 4B), 500 μm (FIG. 4E, FIG. 4F), 200 μm (FIG. 4G, FIG. 4H, FIGS. 4I-L).

FIG. 5 is a series of images and bar graphs showing CCT2/CTGF promotes ex vivo morphogenesis of calvarial suture in organ culture. The interfrontal suture (IFS) of the Sprague Dawley rat was patent by postnatal day 10 (p10) (FIG. 5A), showing fibroblastic soft tissue between mineralized bones. The IFS undergoes ectopic mineralization or synostosis by approximately postnatal day 25 (p25). The representative calvarial suture by p35 was characterized by the virtual disappearance of fibroblastic soft tissue and its replacement by dense, mineralizing tissue between existing mineralized bone (FIG. 5B). Delivery of 50 ng/mL CCN2/CTGF to p10 explant for 25 days rescued the calvarial suture from undergoing ectopic mineralization (FIG. 5C), showing the presence of a fibroblastic, soft tissue interface between mineralizing bone. b: bone, s: suture. FSP1 and vimentin were expressed in the representative p10 innate calvarial suture and the representative CCN2/CTGF-treated calvarial suture (FIG. 5D, FIG. 5F for FSP1) and (FIG. 5G, FIG. 5I for vimentin), in comparison with faint FSP1 expression and virtual absence of vimentin without CCN2/CTGF (FIG. 5E and FIG. 5H, respective). The width of the representative calvarial suture by p35 without CCN2/CTGF delivery was narrow on 3D pCT reconstructed sample (FIG. 5J), in comparison with wide, patent suture with CCN2/CTGF treatment by p35 (FIG. 5K). Quantitatively, the average width of CCN2/CTGF-treated sutures was significantly wider than without CCN2/CTGF (FIG. 5L) (p<0.05; N=8). Harvested soft tissue from CCN2/CTGF-treated sutures showed significantly more Tn-C contents than CCN2/CTGF-free sutures (FIG. 5M) (p<0.05). Scale: 60 μm.

FIG. 6 is a series of images demonstrating that calvarial suture mesenchymal cells showed multi-lineage differentiation capacity. Cells isolated from native, patent calvarial sutures by p7 readily differentiated into fibroblast-like cells that are highly Trichrome positive upon 100 ng/mL CCN2/CTGF stimulation (FIG. 6A). In contrast, isolated calvarial suture cells without CCN2/CTGF treatment continued to assume MSC morphology and synthesized little collagen (FIG. 6E). Suture cells from p7 calvaria that was about to undergo synostosis within 20-30 days readily differentiated into osteoblasts under osteogenic stimulation with or without CCN2/CTGF (FIG. 6B, FIG. 6C), in comparison to isolated cells without osteogenic stimulation (FIG. 6F). Also, isolated calvarial suture cells underwent adipogenic differentiation under permissive conditions (FIG. 6D), in comparison with isolated suture cells without adipogenic stimulation (FIG. 6G). Scale: 100 μm (FIG. 6A, FIGS. 6D-G), 50 μm (FIG. 6B, FIG. 6C).

FIG. 7 is a series of images and bar graphs showing CCN2/CTGF-treated cells are neither osteogenic nor chondrogenic. Von Kossa staining was negative in CCN2/CTGF-treated MSCs (FIG. 7B), just as MSCs without CCN2/CTGF treatment (FIG. 7A). In contrast, MSCs subjected to osteogenic stimulation readily differentiated into osteogenic cells that elaborated minerals (FIG. 7C). Safranin O staining was negative in CCN2/CTGF-treated MSCs (FIG. 7E), just as MSCs without CCN2/CTGF treatment (FIG. 7D). In contrast, MSCs subjected to chondrogenic stimulation readily differentiated into chondrogenic cells that were safranin O positive (FIG. 7F). Quantitatively, MSCs under osteogenic stimulation elaborated significantly more calcium than the same subpopulation of cells with or without CCN2/CTGF treatment (FIG. 7G) (n=5, *:p<0.05, **:p<0.01). In parallel, MSCs under chondrogenic stimulation produced significantly more glycosaminoglycans (GAG) than the same subpopulation of cells with or without CCN2/CTGF treatment (H) (n=5, *:p<0.05, **:p<0.01). Scale: 100 μm.

FIG. 8 is a series of gel images for Phosphor-p38 and Phosphor-TrKA from human bone marrow MSCs treated with CTGF (100 ng/ml).

FIG. 9 is a series of images of human bone marrow MSCs treated with 100 ng/ml CTGF (FIG. 9A); 0.2 μM p38 inhibitor and 100 ng/ml CTGF (FIG. 9B); 1 μM p38 inhibitor and 100 ng/ml CTGF (FIG. 9C); and 5 μM p38 inhibitor and 100 ng/ml CTGF (FIG. 9D).

FIG. 10 is a series of images of human bone marrow MSCs treated with ascorbic acid and 100 ng/ml CTGF (FIG. 10A); ascorbic acid, 0.2 μM p38 inhibitor, and 100 ng/ml CTGF (FIG. 10B); ascorbic acid, 1 μM p38 inhibitor, and 100 ng/ml CTGF (FIG. 100); and ascorbic acid, 5 μM p38 inhibitor, and 100 ng/ml CTGF (FIG. 10D).

FIG. 11 is a series of images of stained human bone marrow MSCs treated with ascorbic acid and 100 ng/ml CTGF (FIG. 11A); ascorbic acid, 50 ng/ml K252a/DMSO, and 100 ng/ml CTGF (FIG. 11B); ascorbic acid, 100 ng/ml K252a/DMSO, and 100 ng/ml CTGF (FIG. 11C); ascorbic acid, 200 ng/ml K252a/DMSO, and 100 ng/ml CTGF (FIG. 11D); ascorbic acid, 500 ng/ml K252a/DMSO, and 100 ng/ml CTGF (FIG. 11E); and control MSC (FIG. 11F).

FIG. 12 is a series of images of stained human bone marrow MSCs treated with ascorbic acid and 100 ng/ml CTGF (FIG. 12A); ascorbic acid, 50 ng/ml K252a/DMSO, and 100 ng/ml CTGF (FIG. 12B); ascorbic acid, 100 ng/ml K252a/DMSO, and 100 ng/ml CTGF (FIG. 12C); ascorbic acid, 200 ng/ml K252a/DMSO, and 100 ng/ml CTGF (FIG. 12D); ascorbic acid, 500 ng/ml K252a/DMSO, and 100 ng/ml CTGF (FIG. 12E); and control MSC (FIG. 12F).

FIG. 13 is a series of images showing that P38 inhibitor attenuates keloid cell matrix synthesis. FIG. 13A shows cells treated with 0 μM P38 inhibitor. FIG. 13B shows cells treated with 0.2 μM P38 inhibitor. FIG. 13C shows cells treated with 1 μM P38 inhibitor. FIG. 13D shows cells treated with 5 μM P38 inhibitor.

FIG. 14 is a series of images showing that K252A inhibitor inhibits keloid cell matrix synthesis. FIG. 14A shows cells treated with DMSO. FIG. 14B shows cells treated with 0 ng/ml K252A. FIG. 14C shows cells treated with 50 ng/ml K252A. FIG. 14D shows cells treated with 100 ng/ml K252A. FIG. 14E shows cells treated with 200 ng/ml K252A. FIG. 14F shows cells treated with 500 ng/ml K252A.

FIG. 15 is a series of images showing 4× magnification of cells of FIG. 14. FIG. 15A shows cells treated with DMSO. FIG. 15B shows cells treated with 0 ng/ml K252A. FIG. 15C shows cells treated with 50 ng/ml K252A. FIG. 15D shows cells treated with 100 ng/ml K252A. FIG. 15E shows cells treated with 200 ng/ml K252A. FIG. 15F shows cells treated with 500 ng/ml K252A.

FIG. 16 is a series of images showing 10× magnification of cells of FIG. 14. FIG. 16A shows cells treated with DMSO. FIG. 16B shows cells treated with 0 ng/ml K252A. FIG. 16C shows cells treated with 50 ng/ml K252A. FIG. 16D shows cells treated with 100 ng/ml K252A. FIG. 16E shows cells treated with 200 ng/ml K252A. FIG. 16F shows cells treated with 500 ng/ml K252A.

### DETAILED DESCRIPTION OF THE INVENTION

Aspects presented herein are based, at least in part, on the discovery of how to derive fibroblasts from mesenchymal cells; specifically, that fibroblasts can be derived from both appendicular bone marrow and calvarial mesenchymal stem cells (MSCs). CCN2/CTGF stimulated MSCs can form fibroblasts. Shown herein is the derivation of FSP1⁺, vimentin⁺, Coll1⁺ and αSMA⁻ fibroblasts from multipotent MSCs.

Thus, CCN2/CTGF can be used to transform progenitor cells, such as mesenchymal stem cells, in wounds into alpha smooth muscle actin negative fibroblasts that participate in normal wound healing with minimal scarring. It has also been discovered that the axis of CCN2/CTGF and TGFβ1 can specify stepwise and distinctive processes of fibroblast commitment, fibrogenesis and fibrosis. TGFβ1 stimulation of CCN2/CTGF derived fibroblasts can form myofibroblasts, which are implicated in cancer stroma, pathological scars, and organ fibrosis including the heart, lungs, kidney and liver. Thus, inhibition of TGFβ1 can decrease or eliminate myofibroblast formation from CCN2/CTGF stimulated MSCs in wounds, where such increased levels of alpha smooth muscle actin negative fibroblasts can participate in wound healing with further reduced scarring. Furthermore, it has been discovered that P38 inhibitors can act as a fibrosis inhibitor. And it has been discovered that tyrosine kinase inhibitors can act as a fibrosis inhibitor.

One aspect is directed towards compositions comprising CCN2/CTGF and a fibrosis inhibitor, such as an inhibitor of TGFβ, a P38 inhibitor, or a tyrosine kinase inhibitor. As shown herein, CCN2/CTGF can stimulate differentiation of mesechymal progenitor cells to fibroblast cells, thereby increasing levels of fibrogenesis and aiding wound healing. Furthermore, an inhibitor of TGFβ can reduce or eliminate fibroblast differentiation to myofibroblasts, which are associated with negative outcomes in the healing process. And P38 inhibitors or tyrosine kinase inhibitors can act as a fibrosis inhibitor. A composition described herein can be used to facilitate or accelerate healing of tissue wounds, especially soft tissue wounds. A composition described herein can be a pharmaceutical composition. A pharmaceutical composition described herein can include a pharmaceutically acceptable carrier or excipient, as described in further detail below. A pharmaceutical composition described herein can be further formulated to contain additional active agents, including but not limited to an antibiotic or an immunosuppressive agent. A pharmaceutical composition can be formulated for various routes of administration, including topical. Such compositions can induce fibrogenesis, facilitate or accelerate wound healing, reduce myofibroblast formation, reduce fibrosis, or reduce scarring, along with other benefits described herein.

Another aspect is directed to systems for wound healing. Such systems can include conventional medical devices for wound healing, such as a bandage, blood stop powder, or suture, that includes a composition described herein. Such a system can amplify the healing effect of the device through inducing fibrogenesis, facilitating or accelerating wound healing, reducing myofibroblast formation, reducing fibrosis, or reducing scarring (e.g., aberrant, keloid, and hypertrophic scars), along with other benefits described herein.

Another aspect is directed to a method of treating a subject. As described briefly above, and in more depth below, CCN2/CTGF can induce fibrogenesis, facilitate or accelerate wound healing, reduce myofibroblast formation, reduce fibrosis, or reduce scarring, along with other benefits described herein. Methods described herein can thus aid healing of tissue wounds such as, acute or chronic wounds; dermal, ligament, or tendon wounds; open or close wounds; incision, laceration, abrasion, puncture, penetration, or gunshot wounds; or split laceration, over stretching, grinding compression, cut laceration, or tearing wounds; or various combinations thereof.

Another aspect is directed to a method of forming an αSMA− fibroblast. Contacting a αSMA−, CD34− mesenchymal progenitor cell and CCN2/CTGF can result in the CCN2/CTGF-stimulated mesenchymal stem cell differentiating into a αSMA−, FSP1+, vimentin+, Coll1+ fibroblast cell. Such a αSMA− fibroblast can be, for example, introduced to a wound site or included in a composition for wound treatment.

Further discussion of these and other aspects and features are provided below.

### CCN2/CTGF

Various embodiments of compositions, methods, systems, and kits described herein include CCN2/CTGF. As shown herein, CCN2/CTGF can stimulate a αSMA− CD34− mesenchymal progenitor cell to differentiate into a αSMA− fibroblast. Also as shown herein, CCN2/CTGF can attenuate multipotent stemness genes, increase synthesis of collagen type I, and stimulate fibroblastic hallmarks including FSP1, vimentin, fibronectin, and tenacin-C. Furthermore, as shown herein, fibroblasts differentiated from CCN2/CTGF stimulation of αSMA− mesenchymal progenitor cells can retain the αSMA− characteristic.

CCN2/CTGF is a 36-38 kDa, cysteine-rich protein of the CCN family. CCN2/CTGF can be included, alone or in combination with other growth factors or agents, in compositions, methods, systems, and kits described herein. In some embodiments, the CCN2/CTGF is human CCN2/CTGF or recombinant human CCN2/CTGF. For example, the CCN2/CTGF can be that corresponding to Accession No. NP_{—}001892, or a variant thereof. CCN2/CTGF is available from a variety of commercial sources (e.g., BioVendor, Chandler, N.C.; synthetic CCN2/CTGF peptide RANCLVQTTEWSACSKT, SynPep Corporation, Dublin, Calif.).

In some embodiments, CCN2/CTGF comprises the polypeptide of SEQ ID NO: 1, or a polypeptide comprising a sequence having at least about 80% sequence identity thereto and CTGF activity. For example, the CCN2/CTGF can comprise a polypeptide comprising a sequence having at least about 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1 and CTGF activity.

CCN2/CTGF can be administered in formulations as, for example, isolated polypeptides or polynucleotides. Polynucleotide compositions of CCN2/CTGF include, but are not limited to, gene therapy vectors harboring polynucleotides encoding CCN2/CTGF. Gene therapy methods require a polynucleotide that codes for CCN2/CTGF operatively linked or associated to a promoter and any other genetic elements necessary for the expression of CCN2/CTGF by the target tissue. Such gene therapy and delivery techniques are known in the art (see e.g., Smyth Templeton (2003) Gene and Cell Therapy, CRC, ISBN 0824741048). Suitable gene therapy vectors include, but are not limited to, gene therapy vectors that do not integrate into the host genome. Alternatively, suitable gene therapy vectors include, but are not limited to, gene therapy vectors that integrate into the host genome.

A CCN2/CTGF polynucleotide can be delivered in plasmid formulations. Plasmid DNA or RNA formulations generally include sequences encoding CCN2/CTGF that are free from any delivery vehicle that acts to assist, promote or facilitate entry into the cell, including viral sequences, viral particles, liposome formulations, lipofectin or precipitating agents and the like. Optionally, gene therapy compositions of the embodiments can be delivered in liposome formulations and lipofectin formulations, which can be prepared by methods well known to those skilled in the art (see e.g., Smyth Templeton (2003) Gene and Cell Therapy, CRC, ISBN 0824741048). Gene therapy vectors can further comprise suitable adenoviral vectors including, but not limited to for example, those described in Curiel and Douglas (2002) Adenoviral Vectors for Gene Therapy, Academic Press, ISBN 0121995046.

CCN2/CTGF transcribable polynucleotide molecule sequences described above can be provided in a construct. Constructs generally include a promoter operably linked to a transcribable polynucleotide molecule for CCN2/CTGF, and variants thereof as discussed above. Promoter selection can allow expression of CCN2/CTGF under a variety of conditions. Promoters can also be selected on the basis of their regulatory features. Examples of such features include enhancement of transcriptional activity and inducibility. The promoter can be an inducible promoter. For example, the promoter can be induced according to temperature, pH, a hormone, a metabolite (e.g., lactose, mannitol, an amino acid), light (e.g., wavelength specific), osmotic potential (e.g., salt induced), a heavy metal, or an antibiotic. Numerous standard inducible promoters will be known to one of skill in the art.

The term “construct” is understood to refer to any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked.

CCN2/CTGF can be delivered to a subject by transforming a host cell to express CCN2/CTGF and introducing the transformed cell into the subject. Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Polypeptide compositions of the fibrogenic agents include, but are not limited to, CCN2/CTGF polypeptides. Polypeptide compositions of the fibrogenic agents include, but are not limited to, isolated full-length proteins, fragments and variants thereof. Polypeptide fragments of the fibrogenic agents can comprise, or alternatively consist of, propeptide forms of the isolated full-length polypeptides. Polypeptide fragments of the growth factor agents can comprise, or alternatively consist of, mature forms of the isolated full-length polypeptides. Also provided are the polynucleotides encoding the propeptide and mature polypeptides of CCN2/CTGF.

Variants of CCN2/CTGF include, but are not limited to, protein variants that are designed to increase the duration of activity of CCN2/CTGF in vivo. For example, a variant fibrogenic agent includes full length CCN2/CTGF proteins or fragments thereof that are conjugated to polyethylene glycol (PEG) moieties to increase their half-life in vivo (also known as pegylation).

CCN2/CTGF can be provided in formulation(s) as fusion proteins. For example, CCN2/CTGF can be a fusion protein with the F_{c} portion of human IgG. As another example, CCN2/CTGF can be hetero- or homodimers or multimers. Examples of fusion proteins include, but are not limited to, ligand fusions between mature CCN2/CTGF polypeptides and the F_{C} portion of human Immunoglobulin G (IgG). Methods of making fusion proteins and constructs encoding the same are well known in the art.

Various embodiments further contemplate the use of polynucleotides and polypeptides, which can promote fibrogenesis or stimulate fibroblastic differentiation, having at least 80% sequence identity to the isolated polynucleotides and polypeptides of CCN2/CTGF described herein. For example, polynucleotides and polypeptides can have at least 81%, 82%, 83%, 84%, 85%, 86%, 87%, 88%, 89%, 90%, 91%, 92%, 93%, 94%, 95%, 96%, 97%, 98%, or 99% sequence identity to the isolated polynucleotides and polypeptides of CCN2/CTGF described herein.

CCN2/CTGF can be administered at concentrations of from about 0.1 ng/ml to about 100 mg/ml. For example, CCN2/CTGF can be administered at concentrations of about 0.1 ng/ml, 1 ng/ml, 10 ng/ml, 100 ng/ml, 1 mg/ml, 10 mg/ml, or 100 mg/ml. As another example, CCN2/CTGF can be administered at concentrations of about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 ng/mL. As another example, CCN2/CTGF can be administered at concentrations of about 50, 100, 150, 200, 250, 300, 350, 400, 450, 500, 550, 600, 700, 750, 800, 850, 900, 950, or 1000 ng/mL. As a further example, CCN2/CTGF can be administered at about 100 ng/ml. A skilled artisan will recognize that such effective amounts can be reflected in the amount of CCN2/CTGF present in a pharmaceutical composition, for example, on a per dosage level.

In some embodiments, CCN2/CTGF can be incorporated into a delivery vehicle. Various delivery vehicles are discussed below. In one embodiment, a CCN2/CTGF-containing composition is encapsulated in polymeric microspheres. CCN2/CTGF can be encapsulated in polymeric microspheres at a ratio of about 100 mg to about 500 mg polymer to about 1 to about 100 μg of CCN2/CTGF. For example, CCN2/CTGF can be encapsulated in polymeric microspheres at a ratio of about 10:10, about 50:10, about 100:10, about 150:10, about 200:10, about 250:10, about 300:10, about 350:10, about 400:10, about 450:10 or about 500:10 mg polymer:μg of CCN2/CTGF. In one embodiment, composition is encapsulated in a polymeric microsphere at a ratio of about 250 mg polymer to about 10 μg of CTGF.

In some embodiments, a CCN2/CTGF-containing composition can be administered to a tissue wound by introducing about 1 to about 100 mg of CTGF-encapsulated microspheres to a a tissue wound. For example, about 10, about 15, about 20, about 25, about 30, about 35, about 40, about 45, about 50, about 55, about 60, about 65, about 76, about 75, about 80, about 85, about 90, about 95, or about 100 mg of CTGF-encapsulated microspheres can be introduced to a tissue wound.

CCN2/CTGF can be administered at a pH of about 3 to 8.

CCN2/CTGF or CCN2/CTGF formulations can be sterile. For example, sterility can be readily accomplished by filtration through sterile filtration membranes (e.g., 0.2 micron membranes or filters).

As one of skill in the art will recognize, the concentrations of CCN2/CTGF, or other fibrogenic agents, can be variable based on the desired length or degree of promotion of fibroblast differentiation or fibrogenesis. Similarly, one of skill in the art will understand that the duration of sustained release can be modified by the manipulation of the compositions comprising the sustained release formulation, such as for example, modifying the percent of biostable polymers found within a sustained release polymer.

Determination of an effective amount of a CCN2/CTGF for purposes described herein is within the ordinary skill of the art.

### MSCs

In some embodiments, the composition, method, system, or kit can include mesenchymal progenitor cells. It has been discovered that fibroblasts can be derived from progenitor cells of mesenchymal origin. As shown herein, CCN2/CTGF can stimulate a CD34−, αSMA− mesenchymal progenitor cell to differentiate into a αSMA− fibroblast cell. Providing a mesenchymal progenitor cells (e.g., a CD34− mesenchymal stem cell) in a composition with CCN2/CTGF can thereby provide for delivery of a αSMA− fibroblast cell to a wound site, where such fibroblast can, for example, increase fibrogenesis and aid wound healing.

As used herein, a mesenchymal progenitor cells or progenitor cell of mesenchymal origin is a CD34⁻ progenitor cell, such as a CD34⁻ mesenchymal stem cell. Generally, the progenitor cells of mesenchymal origin are precursors to fibroblasts and differentiate in the presence of CCN2/CTGF. Fibroblasts can be derived from CD34⁻ progenitor cells. CD34⁻ cells differ substantially from CD34⁺ cells of the hematopoietic lineage (37, 38). Fibroblasts can be derived from αSMA⁻ progenitor cells. Fibroblasts can be derived from CD34⁻, αSMA⁻ progenitor cells. For example, and as demonstrated herein, fibroblasts can be derived from appendicular bone marrow of mesodermal origin and calvarial suture of neural crest origin. In some embodiments, the progenitor cells used as the starting material from which fibroblasts are derived are not CD34+ and αSMA+ fibrocytes, which are thought to migrate from bone marrow to cancer stroma or peripheral wounds (6).

Mesenchymal progenitor cells for differentiation into fibroblasts can be obtained from a variety of sources. Mesenchymal progenitor cells for differentiation into fibroblasts can be autogeneic (i.e., from the same subject), allogeneic (i.e., from a genetically non-identical donor of the same species), isogeneic (i.e., from a genetically identical donor), or xengeneic (i.e., from a different species) to a subject. For example, mesenchymal progenitor cells can be isolated from a subject that is to be treated according to methods described herein.

Mesenchymal progenitor cells can be isolated, purified, and/or cultured by a variety of means known to the art Methods for the isolation and culture of progenitor cells are discussed in, for example, Vunjak-Novakovic and Freshney (2006) Culture of Cells for Tissue Engineering, Wiley-Liss, ISBN 0471629359. For example, MSCs can be isolated from bone marrow. Cell isolation can be through methods generally known in the art, such as bone marrow aspiration. The mesenchymal progenitor cells can be derived from the same or different species as the transplant recipient. For example, the mesenchymal progenitor cells can be derived from an animal, including, but not limited to, mammals, reptiles, and avians, more preferably horses, cows, dogs, cats, sheep, pigs, and chickens, and most preferably human.

### Fibroblasts

Provided herein are CCN2/CTGF derived αSMA− fibroblasts. In some embodiments, CCN2/CTGF derived fibroblasts are FSP1+, vimentin+, Coll1+ and αSMA−. Such fibroblasts can participate in, for example, normal wound healing with minimal scars. As shown herein, CCN2/CTGF can be used to favor fibrogenesis, rather than osteogenesis.

CCN2/CTGF, a 36-38 kDa, cysteine-rich protein of the CCN family, can be used for mesenchymal differentiation into fibroblasts. CCN2/CTGF stimulated MSCs can attenuate multipotent stemness genes. CCN2/CTGF stimulated MSCs can have increased synthesis of collagen type I. CCN2/CTGF stimulated MSCs can express fibroblastic hallmarks including FSP1, vimentin, fibronectin, and tenacin-C. CCN2/CTGF stimulated MSCs can be αSMA negative.

CCN2/CTGF stimulated MSCs can have a stable lineage. For example, CCN2/CTGF-derived fibroblastic cells can have a diminished capacity to differentiate into other mesenchymal non-fibroblastic lineages including osteoblasts, chondrocytes and adipocytes.

According to various embodiments described herein, CCN2/CTGF can be contacted with a mesenchymal progenitor cell to stimulate formation of fibroblasts. Methods of culturing progenitor cells are generally known in the art and such methods can be adapted so as to provide optimal conditions for differentiation of mesenchymal progenitor cells contacted with CCN2/CTGF.

In some embodiments, CCN2/CTGF derived αSMA− fibroblasts are present as an enriched cell culture. For example, a culture of mesenchymal stem cells treated with CCN2/CTGF can contain at least about 10%, at least about 20%, at least about 30%, at least about 40%, at least about 50%, at least about 60%, at least about 70%, at least about 80%, at least about 90%, at least about 95%, or at least about 99% αSMA− fibroblasts.

### TGFB Inhibitors

In some embodiments, compositions, methods, systems, and kits include a TGF inhibitor. As shown herein, while CCN2/CTGF-treated MSCs were αSMA−, further stimulation with TGFβ1 can transform the cells into αSMA+ cells characteristic of myofibroblasts that significantly contracted collagen gel in vitro. A TGFβ inhibitor can prevent formation of myofibroblasts from fibroblasts. A TGFβ inhibitor can inhibit fibrosis.

Fibroblast contraction of granular tissue is necessary for the healing of normal dermal wounds (6). But myofibroblast (a αSMA+ cell type) overpopulation and associated excessive collagen production, disorganization, and excessive wound contraction can lead to pathological dermal wound healing and scarring. αSMA+ myofibroblasts are implicated in invasive tumor (cancer stroma), aberrant dermal healing, pathological scars, and organ fibrosis including the heart, lungs, kidney and liver. By reducing formation of myofibroblasts, once can reduce such negative associated effects.

Thus, inhibition or decreased levels of TGFβ (e.g., TGFβ1) can reduce or eliminate transformation of CCN2/CTGF treated fibroblasts into myofibroblasts, thereby avoiding negative complications associated with αSMA+ myofibroblasts.

A TGFβ inhibitor can be included in the same composition as CCN2/CTGF or another composition. A TGFβ inhibitor and CCN2/CTGF can be administered to a subject consecutively or simultaneously. For example, where a first composition includes both CCN2/CTGF and a TGFβ inhibitor, then administration can be consecutive. As another example, where a first composition includes CCN2/CTGF and a second composition includes a TGFβ inhibitor, then administration can be consecutive. Alternatively, where a first composition includes CCN2/CTGF and a second composition includes a TGFβ inhibitor, administration can be simultaneous. It is understood that CCN2/CTGF or a fibrosis inhibitor (e.g., a TGFβ inhibitor, P38 inhibitor, or tyrosine kinase inhibitor) may be administered independently in addition to a formulation containing both, fpor example, to further adjust amounts of each agent present at a wound site.

In some embodiments, an inhibitor of TGF can reduce or eliminate formation of myofibroblasts from fibroblasts. For example, an inhibitor of TGF can reduce or eliminate formation of αSMA+ myofibroblasts from αSMA− fibroblasts. The inhibitor can be an inhibitor of TGFβ1. The inhibitor can be an inhibitor of TGFβ2. The inhibitor can be an inhibitor of TGFβ3. For example, an inhibitor of TGFβ1 can reduce or eliminate differentiation of αSMA+ myofibroblasts from CCN2/CTGF stimulated αSMA−, CD34-mescnchymal progenitor cells. As another example, an inhibitor of TGFβ1 can reduce or eliminate differentiation of αSMA+ myofibroblasts from αSMA− fibroblasts.

In some embodiments, an inhibitor of TGF can reduce formation of αSMA+ myofibroblasts from αSMA− fibroblasts by about 5%. For example, an inhibitor of TGF can reduce formation of αSMA+ myofibroblasts from αSMA− fibroblasts by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%. In some embodiments, an inhibitor of TGFβ can substantially reduce formation of αSMA+ myofibroblasts from αSMA− fibroblasts.

TGFβ inhibitors (e.g., TGFβ1 inhibitors) for use herein generally include antibodies, small molecules acting as competitive or irreversible antagonists, antisense oligonucleotides, protein aptamers, nucleotide aptamers, and small interfering RNAs.

TGFβ inhibitors for use in compositions and methods described herein can include, but are not limited to, those TGFβ inhibitors described in Saunier and Akhurst 2006 Current Cancer Drug Targets 6(7) 565-578; Callahan et al. 2002 J Med Chem 45(5) 999-1001; and Border et al. 1992 Nature 360, 361-364.

A TGFβ inhibitor for use in compositions and methods described herein can include, but is not limited to, ANG-1122 (Angion Biomedica); AP-11014 (Antisense Pharma); metelimumab (AstraZeneca); fresolimumab (AstraZeneca); mannose-6-phosphate, BTG (BTG); Pharmaprojects No. 6614 (Celgene); NAFB001 (Digna Biotech); NAFB002 (Digna Biotech); TGF-β1 antibody, Lilly (Eli Lilly); LY-2157299 (Eli Lilly); Fetuin (Eli Lilly); TGF-β antagonists, FibroGen (FibroGen); 1D11 (Genzyme); anti-TGFβ MAb-1, Genzyme (Genzyme); SB-431542 (GlaxoSmithKline); activin-like kinase 5 inhibitor, Graceway (Graceway); anti-TGF-β antibodies, Genent (Hoffmann-La Roche); antisense oligonucleotide, II (II-Yang); TGF-β antagonists, Inflazyme (Inflazyme); TGF-β receptor, Insmed (Insmed); TGF-β antagonists, Inspiraplex (Inspiraplex); decorin, Telios (Integra LifeSciences); SX-007 (Johnson & Johnson); TGF-β receptor inhibs, J&J (Johnson & Johnson); TGF-β vaccine, Neovacs (Neovacs); ADMP-1 (NIH); TGF-β antibodies, Manchester; TGF-β antagonists, Sydney; mannose-6-phosphonate, Renovo (Renovo); cancer gene therapy, Resver (Resverlogix); TGF-Beta Shield (Resverlogix); IN-1130 (SK Chemicals); LF-984 (Solvay); TGF-β inhibitors, Mill (Takeda); decorin; and SB-431542 (Sigma);

Other TGFβ inhibitors for use in compositions and methods described herein can be obtained from sources known in the art (e.g., Angion Biomedica; Antisense Pharma; AstraZeneca; AVI BioPharma; Biogen Idec; BTG; Celgene; Digna Biotech; Eli Lilly; FibroGen; Genzyme; GlaxoSmithKline; Graceway; Hoffmann-La Roche; II-Yang; Inflazyme; Insmed; Inspiraplex; Integra LifeSciences; Johnson & Johnson; Neovacs; Renovo; Resverlogix; Solvay; Takeda; and Sigma.

Determination of an effective amount of a TGFβ inhibitor for purposes described herein is within the ordinary skill of the art.

### Fibrosis Inhibitor

In some embodiments, compositions, methods, systems, and kits include a fibrosis inhibitor. It has been discovered that a P38 mitogen-activated protein kinases inhibitor or a tyrosine kinase inhibitor can inhibit fibrosis.

A p38 inhibitor can attenuate fibroblast differentiation from mesenchymal progenitor cells; attenuate keloid cell collagen synthesis; attenuate keloid cell growth, or a combination thereof. Examples of p38 inhibitors include, but are not limited to, Tocriset, SD282, SB239063, SB203580, SB220025, SKF86002, PD169316, SB202190, SC68376, VX702, VX745, R130823, AMG548, BIRB796, SCIO469, SCIO323, FR167653, MW012069ASRM, SD169, RWJ67657, and ARRY797.

Administration of a composition or formulation including a p38 inhibitor can inhibit formation of connective tissue. In some embodiments, a composition or formulation including a p38 inhibitor is contacted with a mesenchymal progenitor cell.

A tyrosine kinase inhibitor can attenuate fibroblast differentiation from mesenchymal progenitor cells; attenuate keloid cell collagen synthesis; attenuate keloid cell growth, or a combination thereof. Examples of tyrosine kinase inhibitor include, but are not limited to, K252a, AG013736 (Axitinib), SKI-606 (Bosutinib), AZD2171 (Cediranib), BMS-354825 (Dasatinib), OSI-774 (Erlotinib), ZD1839 (Gefitinib), STI-571 (Imatinib), GW572016 (Lapatinib), CEP-701 (Lestaurtinib), AMN₁₀₇ (Nilotinib), SU5416 (Semaxanib), SU11248 (Sunitinib), Toceranib, ZD6474 (Vandetanib), Vatalanib (PTK787), ZD 1839, CI-1033, OSI-774, GW 2016, EKB-569, IMC-C225, MDX-447, PKI 116, ABX-EGF, AG-82, AG-18, AG-490, AG-17, AG-213, AG-494, AG-825, AG-879, AG-1112, AG-1296, AG-1478, AG-126, RG-13022, RG-14620, and AG-555. Administration of a composition or formulation including a tyrosine kinase inhibitor can inhibit formation of connective tissue. In some embodiments, a composition or formulation including a tyrosine kinase inhibitor is contacted with a mesenchymal progenitor cell.

### Formulation and Carriers

In various embodiments, compositions, methods, systems, and kits include a formulated composition. The compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, preferably in purified form, together with a suitable amount of carrier so as to provide the form for proper administration to the subject.

The formulation should suit the mode of administration. The agents of use with the current invention can be formulated by known methods for administration to a subject using several routes which include, but are not limited to, topical, parenteral, pulmonary, oral, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Compositions and formulations described herein can optionally include antibiotics that may be co-administered so as to prevent infection by obligate or opportunistic pathogens that are introduced to the subject during the procedure. Antibiotics useful with the growth factor formulations include, but are not limited to, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin.

Compositions and formulations described herein can optionally further include immunosuppressive agents. Suitable immunosuppressive agents that may be administered in combination with the growth factor formulations include, but are not limited to, steroids, cyclosporine, cyclosporine analogs, cyclophosphamide, methylprednisone, prednisone, azathioprine, FK-506, 15-deoxyspergualin, and other immunosuppressive agents that act by suppressing the function of responding T cells. Other immunosuppressive agents that may be administered in combination with the growth factor formulations include, but are not limited to, prednisolone, methotrexate, thalidomide, methoxsalen, rapamycin, leflunomide, mizoribine (Bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

Compositions and formulations described herein can optionally further include a carrier vehicle such as water, saline, Ringer's solution, calcium phosphate based carriers, or dextrose solution. Non-aqueous vehicles such as fixed oils and ethyl oleate are also useful herein, as well as liposomes.

Compositions and formulations described herein can further optionally include substances that enhance isotonicity and chemical stability. Such materials are non-toxic to subjects at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Compositions and formulations described herein can be made available as immediate release formulations, sustained release formulations, or both. One of skill in the art could determine whether a subject would benefit from immediate release formulations or sustained release formulations.

Immediate release formulations include liquid formulations comprising an agent, such as CCN2/CTGF, applied to target area. The liquid formulations can provide CCN2/CTGF in bioavailable form at rates dictated by the fluid properties of the liquid formulation, such as diffusion rates at the site of application, the influence of endogenous fluids, etc. Examples of suitable liquid formulations comprise water, saline, or other acceptable fluid mediums that will not induce host immune responses. Skilled artisans recognize that CCN2/CTGF needs to reside at the situs of defect long enough to promote fibrogenesis, and preferably should not seep to surrounding areas. Using the guidelines provided herein, those skilled in the art are capable of designing a suitable formulation for delivery.

Compositions and formulations described herein, including those containing CCN2/CTGF, can be encapsulated and administered in a variety of carrier delivery systems. The carrier material can contain, be coated with, or infused with a compositions and formulations described herein, for example, a CCN2/CTGF-containing composition or formulation. Examples of carrier materials that can be used in such fashion include, but are not limited to, polymeric delivery systems (e.g., biodegradable polymer material, collagen sponge).

As shown herein, control-released CCN2/CTGF from biocompatible microspheres restored the morphogenesis of a mesenchymal/fibrogenic calvarial suture in vivo that otherwise was destined to undergo ectopic mineralization. In other words, microencapsulated CCN2/CTGF prompted postnatal connective tissue to undergo fibrogenesis in vivo, rather than ectopic mineralization.

Controlled release formulations can contain CCN2/CTGF and other agents (e.g., a TGFβ inhibitor) along with a carrier delivery system. The duration of release from the sustained release formulations is dictated by the nature of the formulation and other factors, such as for example, proximity to bodily fluids, as well as density of application of the formulations, degradation rates of biodegradeable polymers, and other factors. However, sustained release formulations can be designed to provide CCN2/CTGF (and other agents such as a TGFβ inhibitor) in the formulations at relatively consistent concentrations in bioavailable form over extended periods of time.

The carrier delivery system generally encapsulates an active agent, such as CCN2/CTGF, and provides controlled release of the agent over extended periods of time. Generally a carrier includes molecules conjugated to, mixed with, or used for encapsulating an active agent, such as CCN2/CTGF. Carrier-based systems for biomolecular agent delivery can: tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; and/or improve shelf life of the product.

Polymeric release systems can be used to deliver compositions or formulations described herein (see Whittlesey and Shea (2004) Experimental Neurology 190, 1-16). Polymeric systems can also be designed to deliver multiple biomolecules that can act synergistically or sequentially on cellular processes. Polymeric delivery systems can maintain therapeutic levels of growth factors, such as CCN2/CTGF, described herein, reduce harmful side effects, decrease the amount of biomolecule required, decrease the number of dosages, and facilitate delivery of agents with short in vivo half-lives. Release rates can be controlled by altering the pore size, structure, and polymer contents of synthetic polymers such as the nondegradable synthetic polymer EVAc and the degradable synthetic polymer polyester PLGA. Furthermore, the degradation of the material itself serves to govern release profiles, providing an additional level of control over release rate. Polymeric delivery systems described herein can be tailored for release durations of, for example, minutes, hours, days, weeks, and even years depending upon the physical and chemical properties of the delivered molecule, the polymer employed, and the processing conditions used during fabrication.

Both natural (e.g., collagen) and synthetic polymers (e.g., silicone, poly-lactide-co-glycolide (PLGA), and polyethylene vinyl-co-acetate (EVAc)) can be utilized for the local delivery of CCN2/CTGF. Biodegradable polymers are preferable for biomolecule delivery because the device can disappear over time, eliminating the need for surgical retrieval. PLGA is a widely used biopolymer due to its commercial availability, controllable degradation rate, proven biocompatibility, and FDA approval (see e.g., Lu et al. (2000) Biomaterials 21, 1837-1845). Polyanhydrides are a similar class of degradeable polymer that can be used for biomolecule delivery.

Polymeric microspheres can facilitate delivery of compositions or formulations described herein, including CCN2/CTGF or other agents such as a TGFβ inhibitor. For example, sustained delivery microspheres can be stereotactically injected to release a polypeptide or polynucleotide of the growth factor at a target site (e.g., a wound or cranial suture). Microspheres can be produced using naturally occurring or synthetic polymers to produce particulate systems in the size range of 0.1 to 500 μm. Generally, microspheres are physically and chemically more stable than liposomes and allow for higher agent loading. Polymeric micelles and polymeromes are polymeric delivery vehicles with similar characteristics to microspheres and can also facilitate encapsulation and delivery of agents, such as CCN2/CTGF or a fibrosis inhibitor (e.g., a TGFβ inhibitor, P38 inhibitor, or tyrosine kinase inhibitor), described herein. Fabrication, encapsulation, and stabilization of microspheres for biomolecular payloads such as of CCN2/CTGF or a fibrosis inhibitor (e.g., a TGFβ inhibitor, P38 inhibitor, or tyrosine kinase inhibitor), are within the skill of the art (see e.g., Varde & Pack (2004) Expert Opin. Biol. 4(1)35-51). Polymer materials useful for forming microspheres include PLA, PLGA, PLGA coated with DPPC, DPPC, DSPC, EVAc, gelatin, albumin, chitosan, dextran, DL-PLG, SDLMs, PEG (e.g., ProMaxx), sodium hyaluronate, diketopiperazine derivatives (e.g., Technosphere), calcium phosphate-PEG particles, and oligosaccharide derivative DPPG (e.g., Solidose). Encapsulation can be accomplished, for example, using a water/oil single emulsion method, a water-oil-water double emulsion method, or lyophilization. Several commercial encapsulation technologies are available (e.g., ProLease®, Alkerme). Release rate of microspheres can be tailored by type of polymer, polymer molecular weight, copolymer composition, excipients added to the microsphere formulation, and microsphere size.

Polymeric hydrogels, composed of hydrophillic polymers such as collagen, fibrin, and alginate, can also be used for the sustained release of incorporated compositions or formulations, including CCN2/CTGF or a fibrosis inhibitor (e.g., a TGFβ inhibitor, P38 inhibitor, or tyrosine kinase inhibitor) (see e.g., Sakiyama et al. (2001) FASEB J. 15, 1300-1302). Biomolecules incorporated into the hydrogel can stimulate cellular function directly from the matrix or following release.

Three-dimensional polymeric implants, on the millimeter to centimeter scale, can be loaded with compositions or formulations, including CCN2/CTGF or a fibrosis inhibitor (e.g., a TGFβ inhibitor, P38 inhibitor, or tyrosine kinase inhibitor) (see e.g., Teng et al (2002) Proc. Natl. Acad. Sci. U.S.A. 99, 3024-3029). These polymeric implants can serve a structural function for cell adhesion while also providing controlled release of biomolecules. A polymeric implant typically provides a larger depot of the bioactive factor. The implants can also be fabricated into structural supports, tailoring the geometry (e.g., shape, size, porosity) to the application (e.g., conforming to the wound or cranial suture). Three-dimensional polymeric implants for biomolecule delivery can be formulated in a variety of means known to the art including, but not limited to, emulsion methods, solvent casting, and carbon dioxide foaming process (see e.g., Whittlesey and Shea (2004) Experimental Neurology 190, 1-16). Implantable matrix-based delivery systems are also commercially available in a variety of sizes and delivery profiles (e.g., Innovative Research of America, Sarasota, Fla.).

As an alternative to release, compositions or formulations, including CCN2/CTGF or a fibrosis inhibitor (e.g., a TGFβ inhibitor, P38 inhibitor, or tyrosine kinase inhibitor), can be immobilized on or in polymeric delivery systems. This approach includes substrate mediated delivery and solid-phase delivery. Generally, the polymeric substrate functions to support cell adhesion and place the biomolecular cargo directly in the cellular microenvironment (see e.g., Whittlesey and Shea (2004) Experimental Neurology 190, 1-16). Substrate mediated delivery can be used to deliver both nonviral and viral vectors. This approach is especially preferable for viral vector delivery as it mimics how many such vectors associate with the extracellular matrix as a means to facilitate cellular binding and internalization. For example, implantation of an adenovirus-modified collagen gel can result in transduction throughout the matrix with a differing delivery profile as compared to direct injection, thus localizing gene delivery and avoiding distal side effects (see e.g., Levy et al. (2001) Gene Ther. 8, 659-667).

Liposome can be used to facilitate the delivery of compositions or formulations, including CCN2/CTGF or a fibrosis inhibitor (e.g., a TGFβ0 inhibitor, P38 inhibitor, or tyrosine kinase inhibitor), to the target site. The drug carrying capacity and release rate of liposomes can depend on the lipid composition, size, charge, drug/lipid ratio, and method of delivery. Conventional liposomes are composed of neutral or anionic lipids (natural or synthetic). Commonly used lipids are lecithins such as (phosphatidylcholines), phosphatidylethanolamines (PE), sphingomyelins, phosphatidylserines, phosphatidylglycerols (PG), and phosphatidylinositols (PI). A commonly used method of encapsulation is rehydration of a lipid film with a biomolecule solution followed by freeze-thawing and extrusion. Other techniques for forming biomolecule liposomes include the proliposome technique (see e.g., Galovic et al. (2002) Eur. J. Pharm. Sci. 15, 441-448) and the crossflow injection technique (see e.g., Wagner et al. (2002) J. Liposome Res. 12, 259-270). Liposome encapsulation efficiency can be monitored and optimized through various procedures known to the art, including differential scanning calorimetry (see e.g., Lo et al. (199%) J. Pharm. Sci. 84, 805-814).

Excipients can be added to the delivery system to stabilize the emulsion during fabrication and to stabilize the growth factors during fabrication and/or release. In the case of encapsulated proteins such as CCN2/CTGF, addition of excipients, such as PEG, carbohydrates, and buffering salts (e.g., magnesium hydroxide), can prevent aggregation and stabilize the folded protein structure. As another example, encapsulated protein biomolecules in PLGA microspheres in the presence of the hydrophilic excipient mannitol can enhance biomolecular stability. Excipients can also impact release rate. For example, PVA in the biomolecule solution can stabilize the primary emulsion and provide more uniform distribution throughout the matrix, prevent coalescence of inner aqueous-phase droplets, and decrease initial release burst and overall release rate. Coating of microspheres can be used to alter in vivo properties. For example, coating PLGA microspheres with DPPC can decrease uptake of the biomolecule cargo into macrophages. As another example, coating particles with mucoadhesive polymers such as chitosan and hydroxypropylcellulose can increase residency time of carriers.

Liquid compositions that are useful for the delivery of growth factor formulations in vivo include conjugates of CCN2/CTGF with a water-insoluble biocompatible polymer, with the dissolution of the resultant polymer-active agent conjugate in a biocompatible solvent to form a liquid polymer system. In addition, the liquid polymer system may also include a water-insoluble biocompatible polymer that is not conjugated to CCN2/CTGF. In one embodiment, these liquid compositions may be introduced into the body of a subject in liquid form. The liquid composition then solidifies or coagulates in situ to form a controlled release implant where the growth factors are conjugated to the solid matrix polymer.

In one embodiment, the carrier material is provided without growth factor formulations incorporated within the carrier material. In this embodiment, the growth factor formulations are introduced into the carrier material prior to implantation of the material in a subject. In such a situation, agents, such as CCN2/CTGF or a fibrosis inhibitor (e.g., a TGFβ inhibitor, P38 inhibitor, or tyrosine kinase inhibitor), are placed into a container having a sterile access port, for example, an intravenous solution bag or vial having a stopper pierceable by a hypodermic injection needle. In one embodiment, agents and prepared formulations are stored in separate containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous CCN2/CTGF solution, and the resulting mixture is lyophilized. The fibrogenic agent is prepared by reconstituting the lyophilized agent prior to administration in an appropriate solution, admixed with the prepared CCN2/CTGF formulations and administered to the surface of the carrier material or infused into the carrier material prior to or concurrent with implantation into a subject. Application may be achieved by, for example, immersion of the carrier material in CCN2/CTGF formulations, by spraying CCN2/CTGF formulations on the surface of the carrier material, or by any other means of application.

Agents described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

One aspect provides for a method of treating a wound in a subject by administering compositions described herein to the subject in need thereof. For example, a wound can be treated by administering a CCN2/CTGF composition to a subject in need thereof. As another example, a wound can be treated by administering CCN2/CTGF derived αSMA− fibroblasts to a subject in need thereof. As another example, a wound can be treated by administering CCN2/CTGF derived αSMA− fibroblasts and a CCN2/CTGF composition to a subject in need thereof. As another example, a wound can be treated by administering mesenchymal progenitor cells and a CCN2/CTGF composition to a subject in need thereof.

CCN2/CTGF compositions, including those with a TGFβ inhibitor, a P38 inhibtior, or a tyrosine kinase inhibitor, can be according to those described above.

Mesenchymal progenitor cells for differentiation into fibroblasts or administration to a subject, or both, can be autogeneic (i.e., from the same subject), allogeneic (i.e., from a genetically non-identical donor of the same species), isogeneic (i.e., from a genetically identical donor), or xengeneic (i.e., from a different species) to a subject. For example, mesenchymal progenitor cells, or αSMA− fibroblasts derived therefrom, can be admininstered to the same subject from which the mesenchymal progenitor cells were isolated.

In some embodiments, a subject is administered a therapeutically effective amount of CCN2/CTGF, so as to promote fibrogenesis. Some embodiments provide a method for enhancing wound healing. Some embodiments provide a method for regenerating tendon and ligament. Some embodiments provide a method for reducing scarring (e.g., aberrant, keloid, and hypertrophic scars) during the normal healing process. An important distinction exists between fibrogenesis, which represents a normal wound healing process, and fibrosis, which represents aberrant scarring. Provided herein are therapeutic methods which favor fibrogenesis and normal wound healing over fibrosis and aberrant scarring. As shown herein fibroblastic differentiation, fibrogenesis, and fibrosis are three distinctive processes.

Mesenchymally derived fibroblasts can act as repair cells for ligament and tendon injuries. Such injuries have few effective conventional therapies (13, 14). While tendons can harbor multipotent stem/progenitor cells that differentiate into typical mesenchymal lineages including adipose, bone, and cartilage cells (25), these progenitor cells tend to scarify normal tendons when used as an autologous cell source for tissue repair. In contrast, CCN2/CTGF stimulated MSCs can differentiate into αSMA− fibroblasts, which are not associated with fibrosis or excess scarring.

Mesenchymally derived fibroblasts can facilitate tendon and ligament regeneration.

Methods described herein are generally performed on a subject in need thereof. A subject in need of the therapeutic methods described herein can be diagnosed with a wound. A determination of the need for treatment will typically be assessed by a history and physical exam consistent with the disease or condition at issue. Diagnosis of the various conditions treatable by the methods described herein is within the skill of the art. The subject can be an animal subject, preferably a mammal, more preferably horses, cows, dogs, cats, sheep, pigs, mice, rats, monkeys, guinea pigs, and chickens, and most preferably a human.

Wounds treatable with compositions and methods described herein include, but are not limited to a soft tissue wound. For example, a soft tissue wound can include a dermal wound, adipose wound, muscle wound, a ligament wound, a tendon wound, a vascular wound, a connective tissue wound, a cartilage wound, or some combination thereof. In some embodiments, the soft tissue wound comprises a dermal wound.

Wounds treatable with compositions and methods described herein include, but are not limited a chronic tissue wound or an acute tissue wound. For example, a chronic tissue wound includes a chronic soft tissue wound. As another example, an acute tissue wound includes an acute soft tissue wound.

Wounds treatable with compositions and methods described herein include, but are not limited, an open wound or a closed wound. In some embodiments, the tissue wound comprises an open tissue wound. For example, an open tissue wound can include an open wound comprising dermal, ligament, or tendon damage, or some combination thereof. An open wound can be partialy, substantially, or completely closed before, during, or after treatment with a composition or method described herein. Wound closure methods can be any conventional technique, such as steri strips, a cyanoacrylate glue, staples, or sutures. A composition described herein can be coated, suffused, or absorbed into or onto such conventional wound treatment devices, as described further herein.

Wounds treatable with compositions and methods described herein include, but are not limited, an incision wound, a laceration wound (e.g., split laceration, over stretching, grinding compression, cut laceration, or tearing), an abrasion wound, a puncture wound, a penetration wound, or a gunshot wound.

A composition or method described herein can be used before, during, or after a conventional wound treatment approach.

An effective amount of composition or formulation described herein is generally that which can enhance fibroblast differentiation; enhance fibrogenesis; inhibit myofibroblast differentiation; inhibit fibrosis; amplifiy or accelerate healing; or reduce scarring.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a composition comprising CCN2/CTGF (and optionally a TGFβ inhibitor) can be employed in pure form or, where such forms exist, in pharmaceutically acceptable salt form and with or without a pharmaceutically acceptable excipient. For example, the compounds of the invention can be administered, at a reasonable benefit/risk ratio applicable to any medical treatment, in a sufficient amount to increase differentiation of fibroblasts, increase fibrogenesis, or decrease fibrosis.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

The specific therapeutically effective dose level for any particular subject will depend upon a variety of factors including the disorder being treated and the severity of the disorder; activity of the specific compound employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present invention will be decided by an attending physician within the scope of sound medical judgment.

Administration of composition comprising CCN2/CTGF (and optionally a TGFβ inhibitor) can occur as a single event or over a time course of treatment. For example, composition comprising CCN2/CTGF (and optionally a TGFβ inhibitor) can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

CCN2/CTGF can be administered simultaneously with an inhibitor of TGFβ, a P38 inhibitor, or a tyrosine kinase inhibitor. For example, CCN2/CTGF can be administered simultaneously with an inhibitor of TGFβ. Simultaneous administration can occur through administration of separate compositions, each containing one or more of CCN2/CTGF, an inhibitor of TGFβ, a P38 inhibitor, and a tyrosine kinase inhibitor. Simultaneous administration can occur through administration of one composition containing two or more of CCN2/CTGF, an inhibitor of TGFβ, a P38 inhibitor, and a tyrosine kinase inhibitor. CCN2/CTGF can be administered sequentially with an inhibitor of TGFβ, a P38 inhibitor, or a tyrosine kinase inhibitor. For example, CCN2/CTGF can be administered before or after administration of a TGFβ inhibitor.

Treatment in accord with the methods described herein can be performed prior to, concurrent with, or after conventional wound treatment modalities.

### Administration

Compositions described herein can be administered in a variety of means known to the art. The agents can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Compositions comprising an agent described herein can be administered in a variety of methods well known in the arts. Administration can include, for example, methods involving oral injestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 microspheres (e.g., 1-100 μm), reservoir devices, a combination of any of the above, or other suitable delivery vehicles to provide the desired release profile in varying proportions. Other methods of controlled-release delivery of agents will be known to the skilled artisan and are within the scope of the invention.

Delivery systems may include, for example, an infusion pump which may be used to administer the agent in a manner similar to that used for delivering insulin or chemotherapy to specific organs or tumors. Typically, using such a system, the agent(s) is administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems, as discussed in greater detail above. Examples of carrier delivery systems include microspheres, hydrogels, polymeric implants, smart ploymeric carriers, and liposomes (see generally, Uchegbu and Schatzlein, eds. (2006) Polymers in Drug Delivery, CRC, ISBN-10: 0849325331). Carrier-based systems for biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Device and System

In some embodiments, a composition described herein can be coated, suffused, or absorbed into or onto a conventional wound treatment device, such as a drape, bandage, dressing, tape, adhesive layer, splint, blood stop powder, steri strip, cyanoacrylate glue, staple, suture. A material in or on which a composition described herein can be included can be chosen to optimize factors known in the art, such as stemming bleeding, absorbing exudate, easing pain, debriding a wound, controlling moisture content, controlling rate of absorption of a topical medicament, maintaining pH, maintaining temperature, protecting from infection, indicating increased bioburden levels, or promoting healing. A material in or on which a composition described herein can be included can be permaeable or impermeable as desired or necessary for a tissue site. A material in or on which a composition described herein can be included can be a film, gel, foam, paste, granule, or bead. A material in or on which a composition described herein can be included can be in sheet form. A material in or on which a composition described herein can be included can be in a flowable form suitable for pouring or dispensing by other means known in the art. A material in or on which a composition described herein can be included can be in a sprayable form.

A composition described herein can be included in or on a material comprising, for example, polyurethane, polyether, polyester, polyolefin, polyolefin sintered polymer, silicone based compound, acrylic, alginate, hydrocolloid, hydrogel, hydrogel-forming material, polysaccharide, natural fabric, synthetic fabric, polyvinyllchlorides, polyamides, polyethyl eneglycol-polydimethyl diloxan co-polymers, polyphosphazenes, cellulosic polymers, chitosan, PVdF, EVA sintered polymer, PTFE, thermoplastic elastomers (TPE), or combinations thereof, such as polymeric combinations, layered combinations, or both. For example, the drape 125 can comprise an EVA sintered polymer. Commercially available exemplary materials include, but are not limited to, Tyvek (PE), Avery Dennison Med 5625; 3M Ioban2; 3M Steri-Drape 125 2; Nitto Denko Yu-Kiban Perme; 3M Tegaderm; First Water Hydroskin; Opsite; Exopack (a polyurethane film and adhesive); Bayer (a polyurethane film); and DuPont (an etherester film).

Use of such devices or systems comprising a composition described herein can be readily adapted to methods described herein.

### Kits

Also provided are kits. Such kits can include the compositions of the present invention and, in certain embodiments, instructions for administration. Such kits can facilitate performance of the methods described herein. When supplied as a kit, the different components of the composition can be packaged in separate containers and admixed immediately before use. Components include, but are not limited to compositions comprising CCN2/CTGF and a TGFβ inhibitor; and systems and devices described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

### Molecular Engineering

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art. Generally, conservative substitutions can be made at any position so long as the required activity is retained.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity ═X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, TGFβ expressed protein activity can be down-regulated or eliminated using antisense oligonucleotides, protein aptamers, nucleotide aptamers, and RNA interference (RNAi) (e.g., small interfering RNAs (sRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem. Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, Tex.; Sigma Aldrich, Mo.; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinoformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Cell Isolation and Expansion

Human mesenchymal stem cells (hMSCs) were isolated from fresh whole bone marrow samples of two anonymous adult donors (age range: 20-25 yrs old) (AllCells, Berkeley, Calif.). Mononucleated and adherent cells were purified by centrifugation through a density gradient (Ficoll-Paque) per our prior methods (30) and using negative selection following manufacturer's protocols (RosetteSep, StemCell Technologies, Vancouver, Canada) to remove hematopoietic cells and other differentiated cells.

Briefly, bone marrow was transferred to a 50 mL tube, followed by addition of 750 mL of RosetteSep and incubation for 20 min. Then 15 mL PBS in 2% fetal bovine serum (FBS) and 1 mM ethylenediaminetetraacetic acid (EDTA) were added to a total volume of ˜30 mL. The sample was layered on 15 mL Ficoll-Paque and centrifuged 25 min at 3000×g. The entire layer of enriched cells was removed from Ficoll-Paque interface. The cocktail was centrifuged at 1000 rpm for 10 min. Collected cells were counted using trypan blue, plated at 0.5-1 million cells per 100 mm dish and allowed to attach for about 5 days, followed by regular medium change every two days. At 80-90% confluence, cells were trypsinized, centrifuged, resuspended in growth medium as passage 1 (P1) cells, and incubated in 5% CO₂ at 37° C., with fresh medium changes every 3-4 days. Growth medium was defined as Dulbecco's Modified Eagle's Medium-Low Glucose (DMEM-LG; Sigma, St. Louis, Mo.), 1% antibiotic (1× Antibiotic-Antimycotic, including 10 units/L Penicillin G sodium, 10 mg/mL Streptomycin sulfate and 0.25 μg/mL amphotericine B) (Gibco, Invitrogen, Carlsbad, Calif.) and 10% Fetal Bovine Serum (FBS; Atlanta Biologicals, Norcross, Ga.).

To isolate calvarial mesenchymal cells, the posterior interfrontal suture excluding calvarial bones was carefully dissected from p7 rat calvaria using surgical scissors. The overlying periosteum and the underlying dura mater were removed under dissection microscope. The isolated suture mesenchyme was lysed using 2 mg/mL collagenase for 1 hr at 37° C., and filtered with a tissue strainer (Fisher, Pittsburgh, Pa.). After centrifugation, the collected cells were counted using trypan blue, plated at 20,000-30,000 cells per 6-well plate, and allowed to attach for about 5 days, followed by medium change every 2-3 days until confluence. P1 or P2 calvarial cells were induced for osteogenic, chondrogenic, adipogenic, and fibroblastic differentiations for 4 wks as described below.

### Example 2

### Treatment with Connective Tissue Growth Factor

The following example describes selection of CCN2/CTGF concentration for fibroblastic differentiation. Methods are according to Example 1 unless otherwise specified.

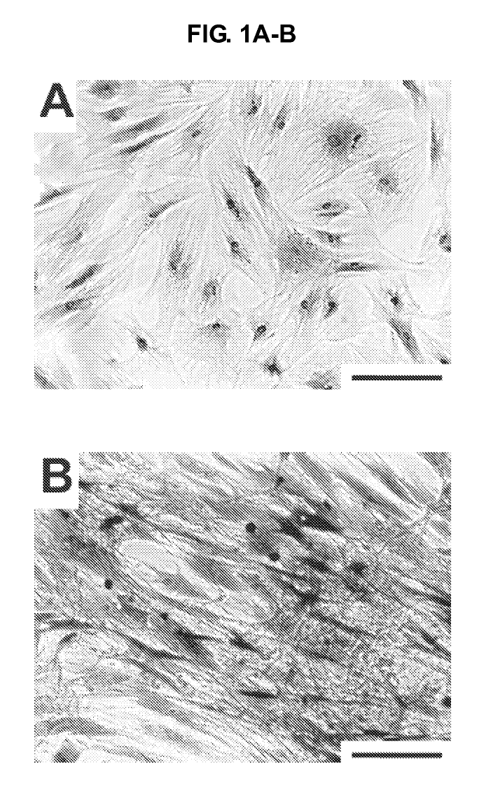

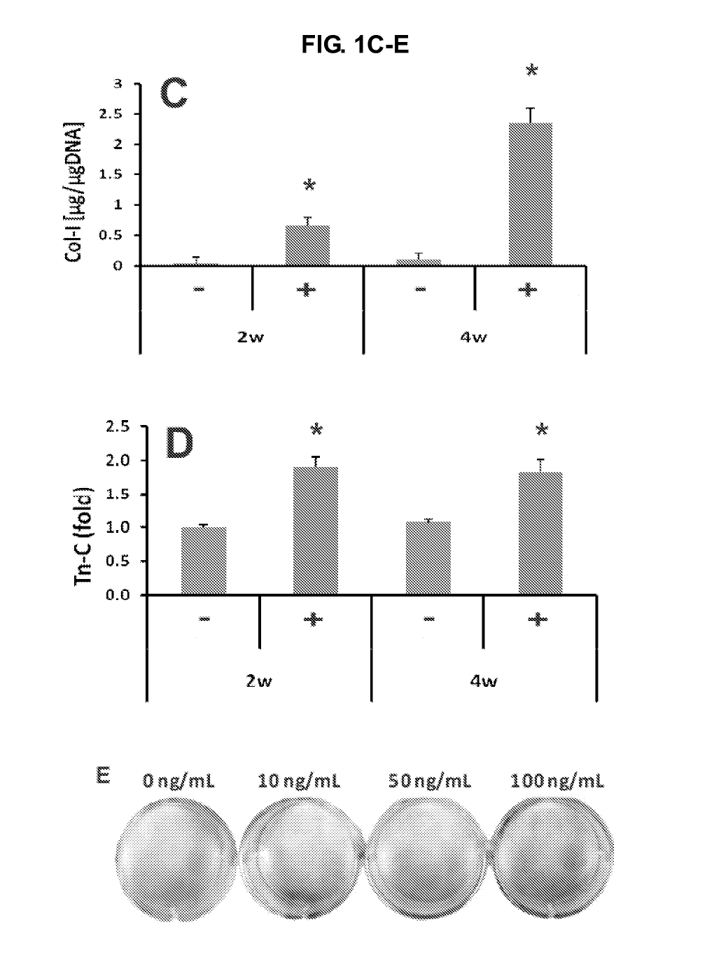

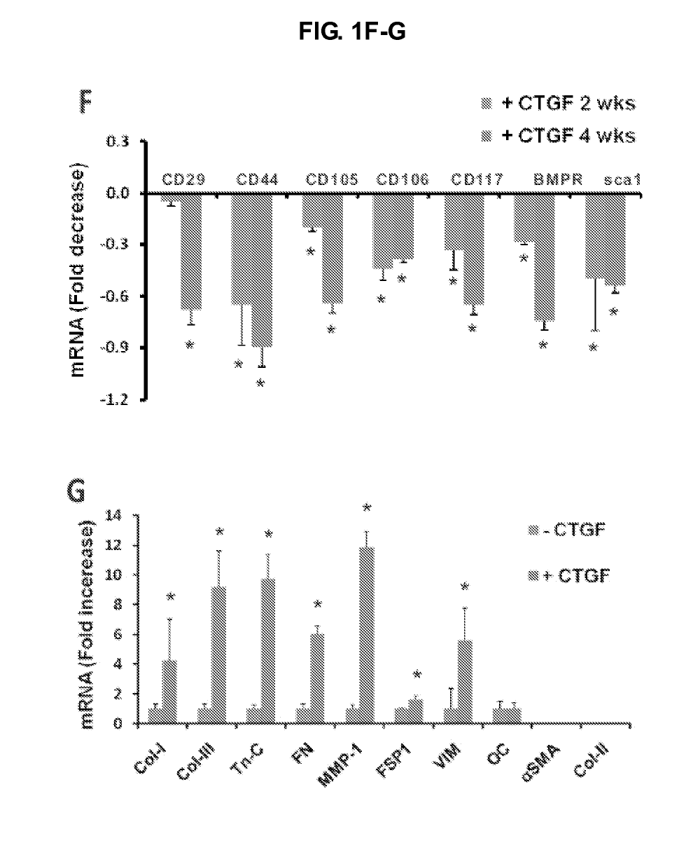

P3 or P4 hMSCs were culture-expanded in monolayer (˜5,000 cells/well) in 12-well plates. At 80-90% confluence, hMSCs were culture-supplemented with 0, 10, 50 or 100 ng/mL recombinant human connective tissue growth factor (CTGF) (BioVendor, Candler, N.C.) and 50 μg/mL ascorbic acid (Sigma), with conditioned medium change every third day. After 4 wks, collagen deposition, as revealed by Goldner's Trichrome staining increased with increasing doses of CCN2/CTGF from 10 to 100 ng/mL (see e.g., FIG. 1E). Accordingly, 100 ng/mL CCN2/CTGF was selected for fibroblastic differentiation for in vitro experiments.

### Example 3

### Collagen Deposition, ELISA and RT-PCR

In this example, collagen deposition was assayed. Methods are according to Examples 1-2 unless otherwise specified.

Two to four weeks following CCN2/CTGF treatment, collagen type I and tenascin-C (Tn-C) were assayed by ELISA using commercial kits (Chondrex, Redmond, Wash. and IBL-America, Minneapolis, Minn.). Collagen type I matrix was lysed using 0.5 M acetic acid. Collagen deposition was visualized using Trichrome staining. Total RNA was isolated using Trizol from CCN2/CTGF-treated hMSCs. Following lysing with Trizol, samples were incubated for 5 min at RT. A total of 0.2 mL chloroform per mL Trizol was added, and followed by mixing and incubation for 3 min. After centrifugation at 12,000×g and 4° C. for 15 min, the upper phase was transferred into a new tube with 500 μL isopropanol added. After 10 min incubation and another centrifugation for 10 min at 12,000×g, the supernatant was discarded. The pellet was washed with 1 mL 75% ethanol and dried for 5-10 min. RNA samples were eluted in 50 μL DEPC-water, assessed for concentration and purity at 260 and 280 nm, and stored at −80° C. prior to reverse transcription. All RNA samples were reverse transcribed using a kit (Applied Biosystems, Foster City, Calif.). For mRNA quantification, real-time quantitative PCR reactions with the cDNA samples were performed using 7300 Real Time PCR System TaqMan® gene expression assays (Applied Biosystems, Foster City, Calif.). Commercially available primers and probes for human collagen types I and III, Tn-C, fibronectin (FN), matrix metallopeptidase-1 (MMP-1), fibroblastic specific protein 1 (FSP1), and vimentin were used (49, 50). Collagen type II (Col-II) and osteocalcin (OC) were selected as chondrogenic and osteogenic differentiation markers, respectively. CD29, CD44, CD105, CD106, CD117, BMPR, and sca1 were selected as MSC markers (30, 31). GAPDH was used as housekeeping gene.

### Example 4

### Osteogenic and Chondrogenic Differentiation

In this example, negative selection experiments were performed to determine whether CCN2/CTGF-treated cells are osteoblasts and chondrocytes. Methods are according to Examples 1-3 unless otherwise specified.

Chondrogenic medium was supplemented with 10 ng/mL transforming growth factor β3 (TGFβ3) (R&D Systems, Minneapolis, Minn.), whereas osteogenic medium was supplemented with 100 nM dexamethasone, 10 mM β-glycerophosphate, and 0.05 mM ascorbic acid-2-phosphate (Sigma) per our prior methods (31, 51). At Days 14 and 28, von Kossa staining and calcium assay was performed to evaluate osteogenic differentiation, whereas safranin O (Saf-O) straining and glycosaminoglycan (GAG) assays were performed to evaluate chondrogenic differentiation (Blyscan™, Biocolor, UK).

### Example 5

### Cloning of hMSCs

In this example, several MSC clones were established by limited dilution. Methods are according to Examples 1-4 unless otherwise specified.

Briefly, P0 hMSCs were suspended at a concentration of 1 cell/200 μL and seeded in 96-well plates with 200 μL medium per well. After 24 hrs, wells with only one cell were marked and maintained in culture at 37° C. and 5% CO2, with medium change twice a week. After 3-4 wks, single-cell derived clones were treated with 0.25% trypsin-EDTA and seeded into 24-well plates for further expansion. Clonal progenies were transferred to 6-well plates upon ˜80% confluence, and maintained with medium change every 3-4 days.

### Example 6

### Further Differentiation of MSC-Derived Fibroblasts

In this example, MSC-derived fibroblasts were subjected to osteogenic, chondrogenic, and adipogenic differentiations. Methods are according to Examples 1-5 unless otherwise specified.

Osteogenic and chondrogenic medium were prepared as described above. Adipogenic medium contained 0.5 mM dexamethasone, 0.5 mM isobutylmethylxanthine, and 50 mM indomethacin per our prior methods (31, 52, 53). In 4 wks, Alizarin Red, Saf-O, and Oil-Red O staining were performed.

### Example 7

### Myofibroblastic Differentiation of MSC-Derived Fibroblasts

This example describes myofibroblastic differentiation of MSC-derived fibroblasts. Methods are according to Examples 1-6 unless otherwise specified.

MSC-derived fibroblasts were subjected to 5 ng/mL recombinant human TGFβ1 (R&D Systems, Minneapolis, Minn.) for 7 days. Undifferentiated hMSCs were treated with TGFβ1 as control. The expression of αSMA (Abcam, Cambridge, Mass.) was evaluated by immunofluorescence with rhodamine-phalloidin (Invitrogen, Carlsbad, Calif.). The number of αSMA+ cells was quantified by flow cytometry.

Briefly, cells were trypsinized, counted, resuspended into 1×106 cells/tube and fixed in 0.01% formaldehyde. Following washing in 0.1% triton, the cells were incubated with αSMA primary antibody (Abcam, Cambridge, Mass.) for 30 min. Following centrifugation at 400 g for 5 min, the cells were incubated with fluorochrome-labeled secondary antibody (Abcam, Cambridge, Mass.) for 30 min in the dark, and resuspended in PBS supplemented with 3% BSA and 1% sodium azide. The samples were then analyzed using a multifunctional cell sorter (FACSAria™ II; BD Biosciences, San Jose, Calif.).

### Example 8

### Collagen Gel Contraction Assay

This example describes collagen gel contraction assays. Methods are according to Examples 1-7 unless otherwise specified.

Neutralized collagen type I (2 mg/mL) (R&D Systems, Minneapolis, Minn.) was mixed thoroughly with 1×10⁶/mL MSCs, MSC-Fb or αSMA+ cells. Cell-populated collagen solution was cast in 24-well plates for 1 hr at 37° C. to induce gelation. Upon 48-hr incubation, cell-populated collagen lattices were physically detached and allowed to float freely in medium. Up to the tested 7 days, the diameters of cell-populated collagen lattices were measured on digital images.

### Example 9

### EX Vivo Modulation of Calvarial Morphogenesis by CCN2/CTGF

This example describes ex vivo modulation of calvarial morphogenesis by CCN2/CTGF. Methods are according to Examples 1-8 unless otherwise specified.

Calvarial explants, including frontal and parietal bones with intervening interfrontal, coronal and sagittal sutures were harvested with intact dura mater from p10 male Sprague-Dawley rats (Harlan, Indianapolis, Ind.). Isolated calvaria were placed in 12-well tissue culture plates with serum-free medium supplemented with 0 or 50 ng/mL recombinant human CCN2/CTGF (35), with medium change every 2 days. The CCN2/CTGF concentration was determined (data not shown). Five to 25 days following CCN2/CTGF treatment, Tn-C contents in supernatant were assayed using ELISA. Explant-cultured calvarial sutures were sectioned sagittally for H&E staining and immunohistochemistry. Calvarial sutures were scanned with pCT (vivaCT 40; Scanco, Southeastern, Pa.) to measure suture width.

### Example 10

### Preparation of CCN2/CTGF-Encapsulated PLGA Microspheres for in Vivo Delivery

This example describes preparation of CCN2/CTGF-encapsulated PLGA microspheres for in vivo delivery. Methods are according to Examples 1-9 unless otherwise specified.

Poly-d-l-lactic-co-glycolic acid (PLGA) microspheres were fabricated by double-emulsion (32, 54). Briefly, a total of 250 mg PLGA was dissolved into 1 mL dichloromethane. Recombinant human CCN2/CTGF (10 μg) was diluted to 50 μL and added to the PLGA solution, forming a mixture (primary emulsion) that was emulsified for 1 min (water-in-oil). The primary emulsion was then added to 2 mL 1% polyvinyl alcohol (PVA, 30,000-70,000 MW), followed by 1 min mixing ([water-in-oil]-in-water). Upon adding 100 mL PVA, the mixture was stirred for 1 min. A total of 100 mL 2% isopropanol was added to the final emulsion and continuously stirred for 2 h to remove the solvent. Control microspheres (empty and without CCN2/CTGF) were fabricated, with the exception of replacing CCN2/CTGF with 50 μL distilled water.

### Example 11

### In Vivo Calvarial Suture Regeneration by Control-Released CCN2/CTGF

This example described in vivo calvarial suture regeneration by control-released CCN2/CTGF. Methods are according to Examples 1-10 unless otherwise specified.

Sprague-Dawley rats develop natively synostosed interfrontal suture by ˜p25 (Moss 1965). Following IACUC approval, rats were anesthetized with 1-5% isoflurane. A 2×4 mm defect was created by resecting the posterior interfrontal suture using a dental bur, with PBS irrigation and care not to damage the underlying dura mater. Collagen sponge (Integra LifeSciences, Plainsboro, N.J.) containing 10 mg CCN2/CTGF-encapsulated PLGA or empty PLGA microspheres was implanted in the surgically created defect. At 4 wks post-op, tissues were harvested and analyzed by μCT. The harvested tissues were sectioned every 4 μm for histology and FSP1 and vimentin immunohistochemistry (Santa Cruz Biotechnology, Santa Cruz, Calif.).

### Example 12

### Data Analysis and Statistics

Methods are according to Examples 1-11 unless otherwise specified. Following confirmation of normal data distribution, one-way analysis of variance (ANOVA) with post-hoc Bonferroni tests were used with p value of 0.05.

### Example 13

### CCN2/CTGF Transforms Mesenchymal Stem Cells into Fibroblastic Cells

Methods are according to Examples 1-12 unless otherwise specified.

Mononuclear and adherent cells were isolated from multiple adult primary human bone marrow samples (30, 31) (see e.g., FIG. 1A). Exposure of 100 ng/mL recombinant human CCN2/CTGF induced remarkable collagen synthesis by 4 wks (see e.g., FIG. 1B), in comparison with MSCs without CCN2/CTGF treatment (see e.g., FIG. 1A). Quantitatively, collagen and tenacin-C synthesis by CCN2/CTGF-treated MSCs was significantly greater at 2 and 4 wks than the same subpopulation of MSCs but without CCN2/CTGF treatment (see e.g., FIG. 1C,D). CCN2/CTGF at 10 ng/mL was sufficient to stimulate collagen synthesis, although 50 ng/mL and 100 ng/mL were apparently more potent (see e.g., 3 panels on the right of FIG. 1E), in comparison with MSCs without CCN2/CTGF (see e.g., left panel in FIG. 1E).

A broad array of multipotent stemness markers associated with MSCs (30) including CD29, CD44, CD105, CD106, CD117, BMPR and Sca1 showed steady decreases over the observed 2 and 4 wks following CCN2/CTGF treatment (see e.g., FIG. 1F; p<0.01). This attenuation of MSC stemness markers was accompanied by concomitant increases of fibroblastic mRNA markers including collagen types I and III, Tn-C, fibronectin, matrix metalloproteinase 1 (MMP-1), fibroblast specific protein 1 (FSP1) and vimentin upon CCN2/CTGF stimulation (see e.g., FIG. 1G). A late stage osteogenic marker, osteopontin and a chondrogenic marker, collagen type II, were undetectable, demonstrating that CCN2/CTGF-treated MSCs were not differentiating into either osteoblasts or chondrocytes, two common mesenchymal lineages.

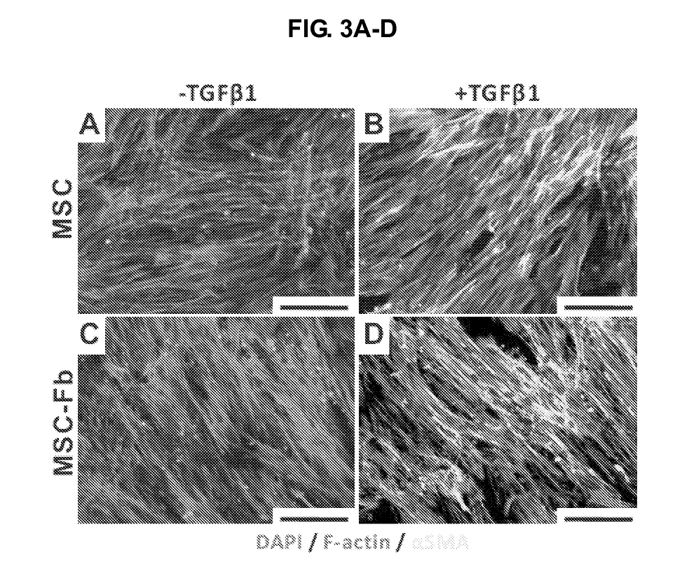

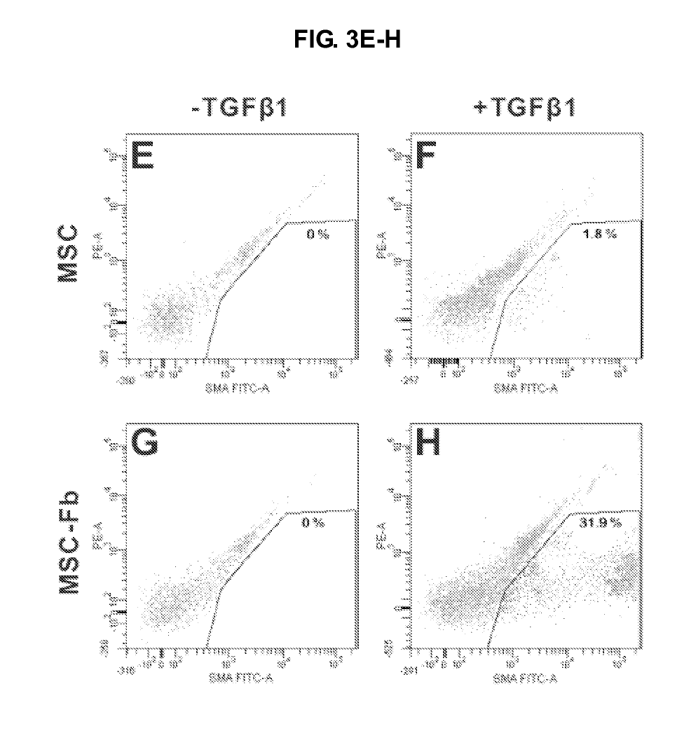

CCN2/CTGF stimulated MSCs remained αSMA negative (see e.g., FIG. 1G, αSMA undetectable), which is confirmed in further experiments (see e.g., FIG. 3A,C) below.

### Example 14

### Attenuated Ability of CCN2/CTGF-Stimulated Mesenchymal Progenitors to Differentiate into Non-Fibroblastic Lineages

Methods are according to Examples 1-13 unless otherwise specified.

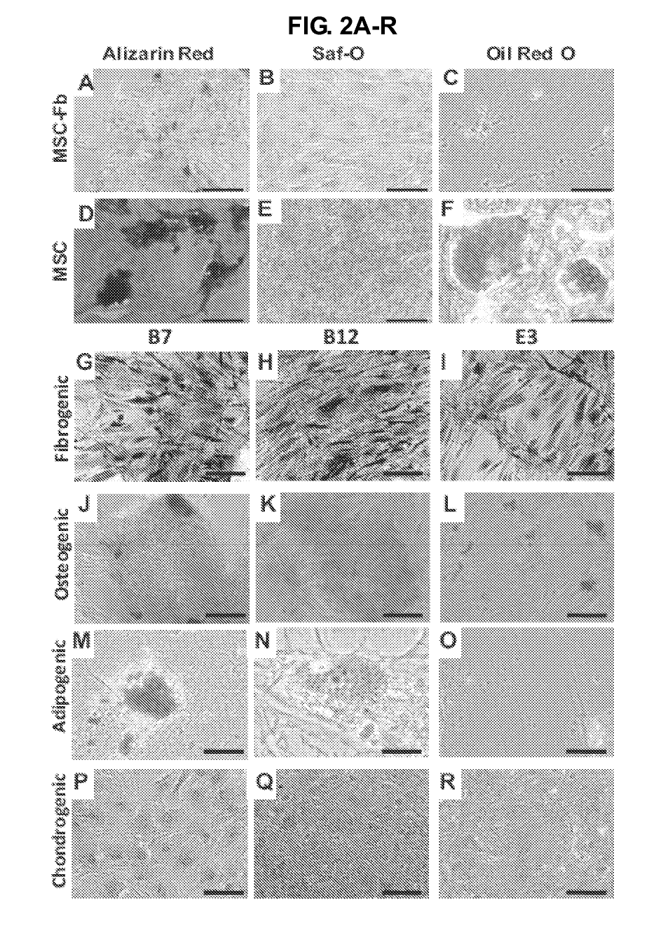

Upon 4 wk CCN2/CTGF stimulation (100 ng/mL), MSCs showed diminished ability to differentiate into osteogenic cells, chondrogenic cells and adipogenic cells (see e.g., FIG. 2A,B,C, respectively), in comparison with CCN2/CTGF-free culture of the same subpopulation of MSCs that readily differentiated into osteoblasts (see e.g., alizarin-red positive cells in FIG. 2D), chondrocytes (see e.g., safranin-O positive cells in FIG. 2E) or adipocytes (see e.g., Oil-red O positive cells in FIG. 2F) under corresponding permissive conditions.

To address a notion that MSC-derived fibroblastic cells in FIG. 1 may have arisen from fibroblasts in the heterogeneous MSC population, clones were isolated from the same subpopulation of MSCs that were studied above (see e.g., FIG. 1). Three tested MSC clones, B7, B12 and E3, readily differentiated into fibroblast-like cells that elaborated collagen (see e.g., FIG. 2G,H,I), osteogenic cells that produced alkaline phosphatase and minerals (see e.g., FIG. 2J,K,L), adipogenic cells that were Oil-red O positive (see e.g., FIG. 2M,N) with the exception of E3 in FIG. 2O, and chondrogenic cells that are safranin O positive (see e.g., FIG. 2P,Q,R). A total of 12 clones isolated, including B7 and B12, differentiated into fibroblastic, osteogenic, chondrogenic, adipogenic cells; whereas a total of 2 clones, including E3, differentiated into fibroblastic, osteogenic and chondrogenic cells but not adipogenic cells. The clonal data show that heterogeneous MSC populations indeed contain multipotent cells that are not end-stage fibroblasts, but are capable of differentiation into common mesenchymal lineages of fibroblasts, osteoblasts, chondrocyte and adipocytes.

Whereas mesenchymal differentiation into osteoblasts, chondrocytes and adipocytes is common practice, fibroblastic differentiation as illustrated herein has not been previously reported.

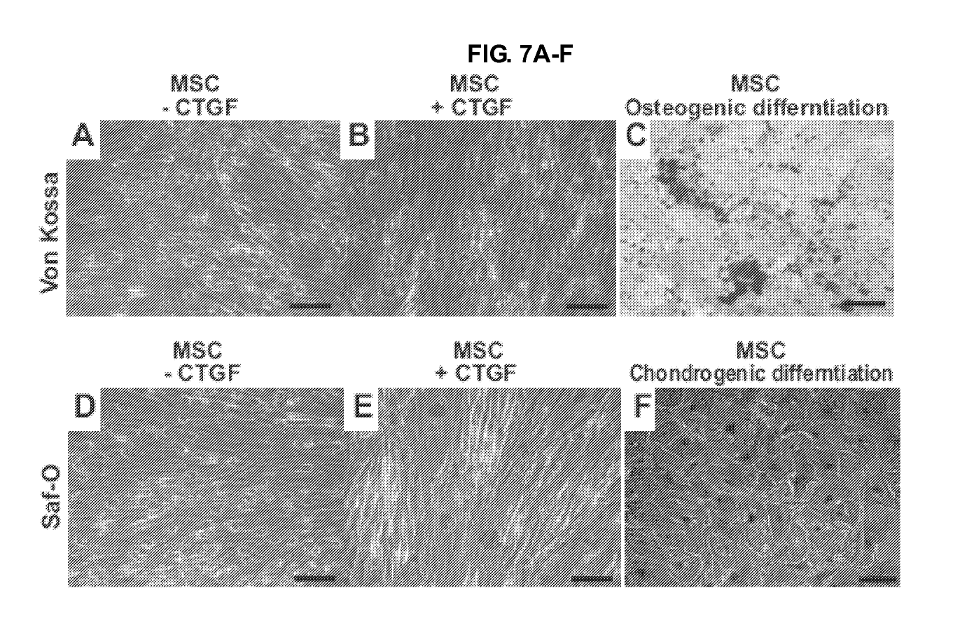

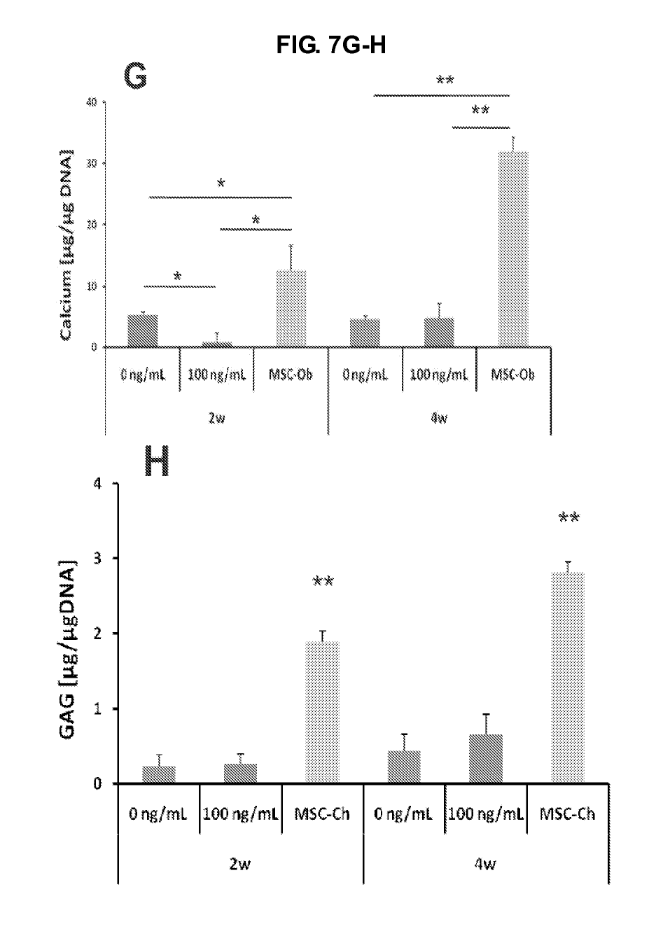

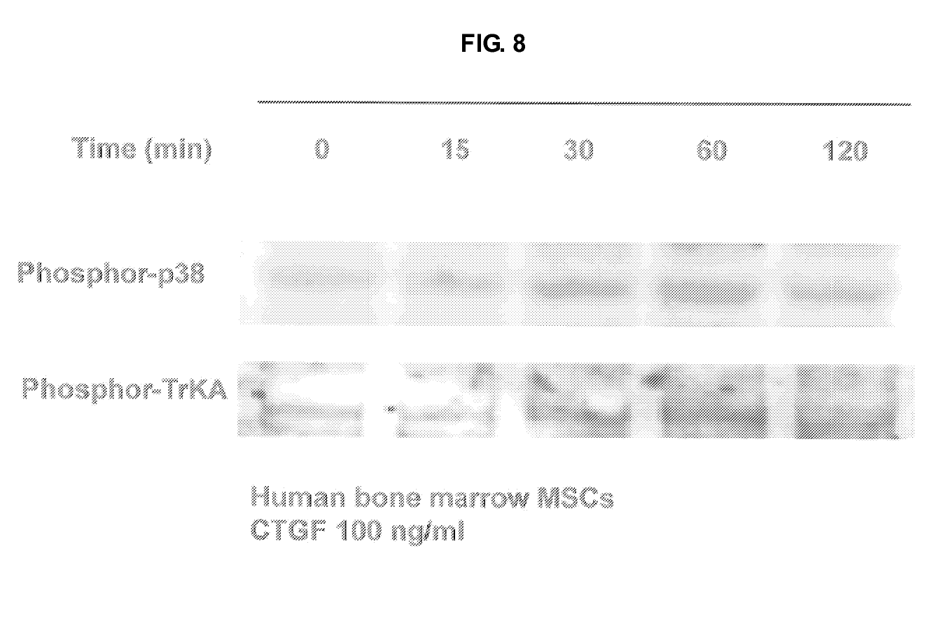

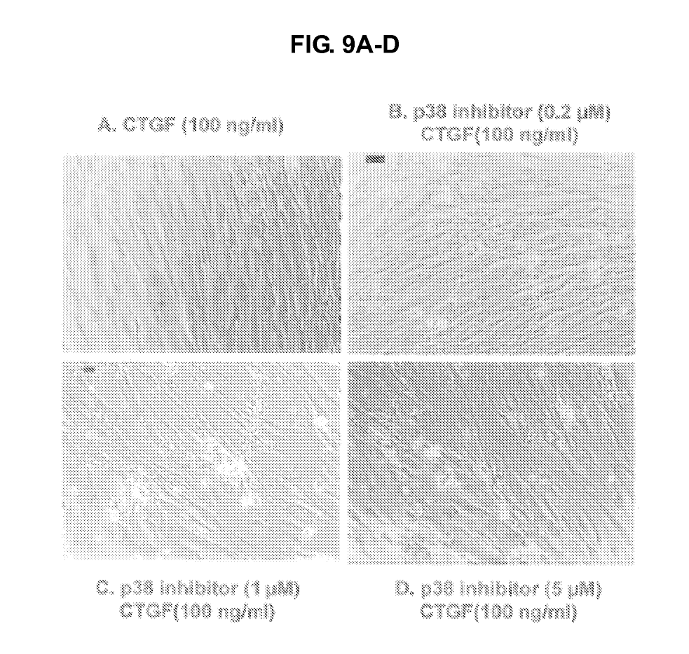

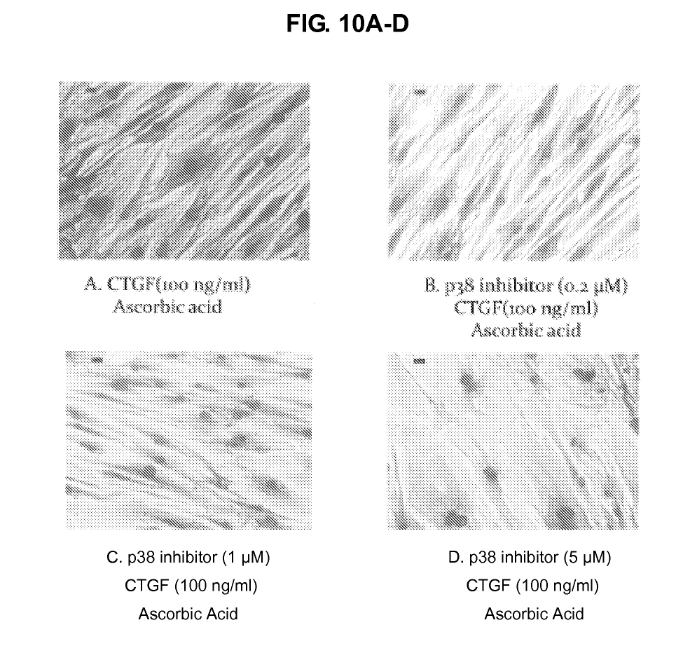

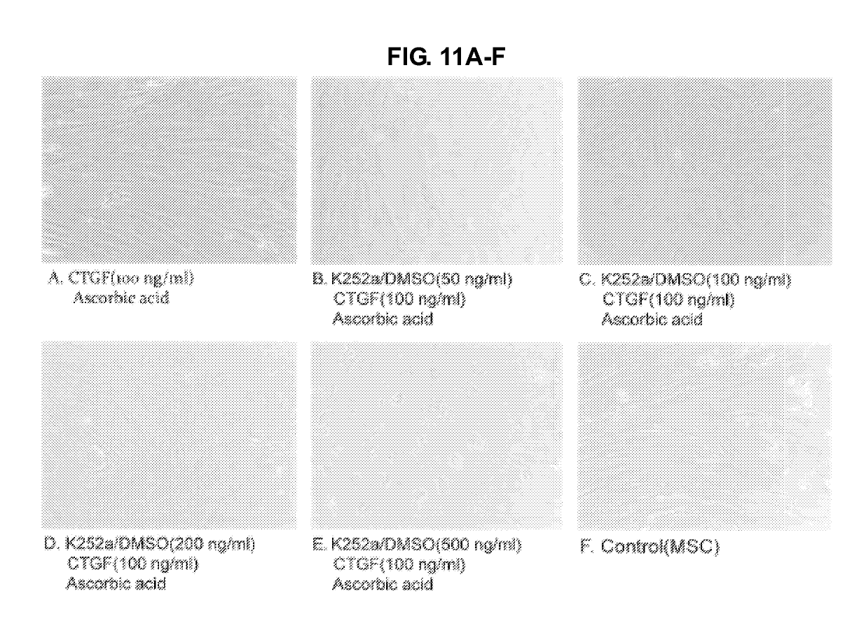

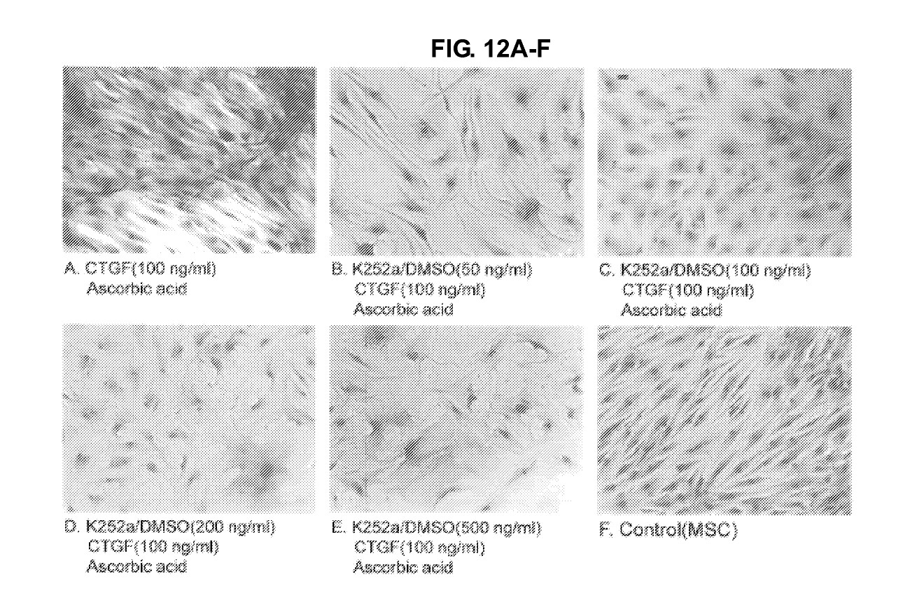

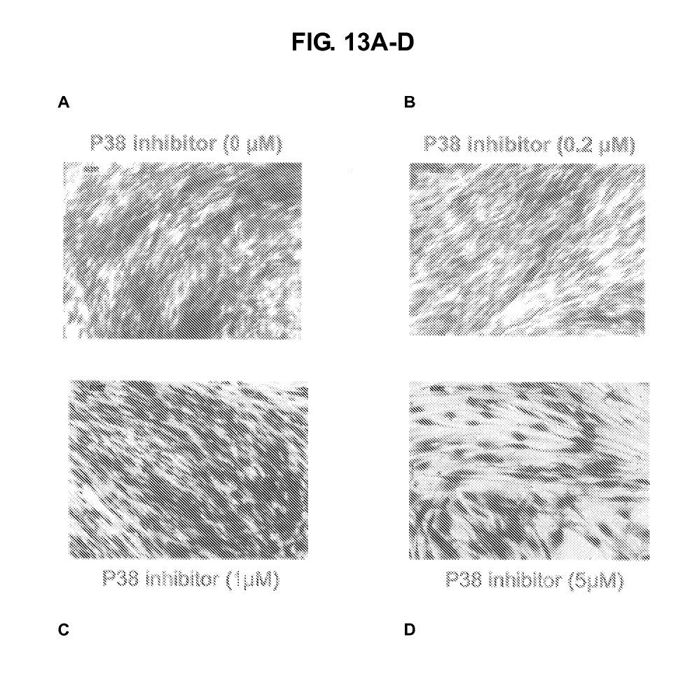

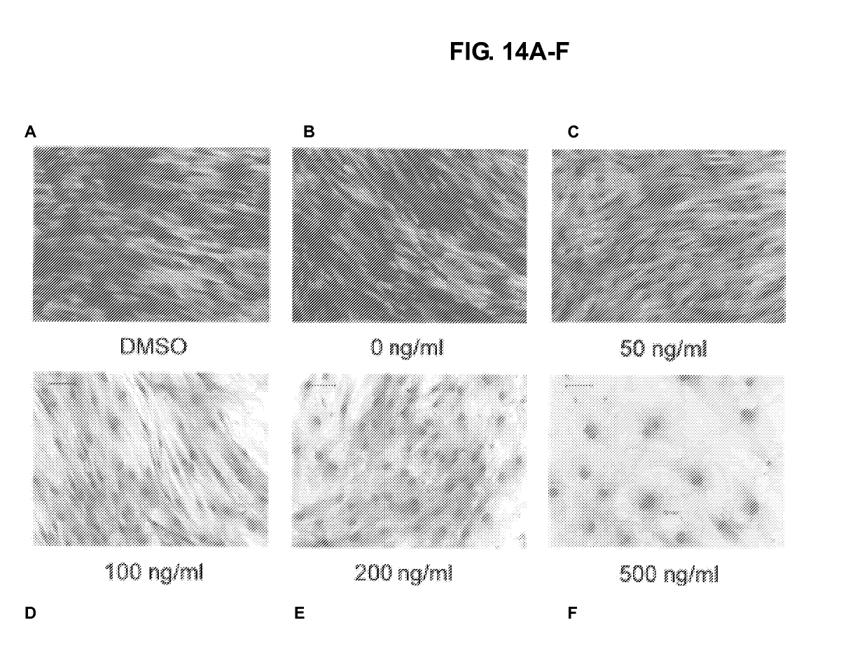

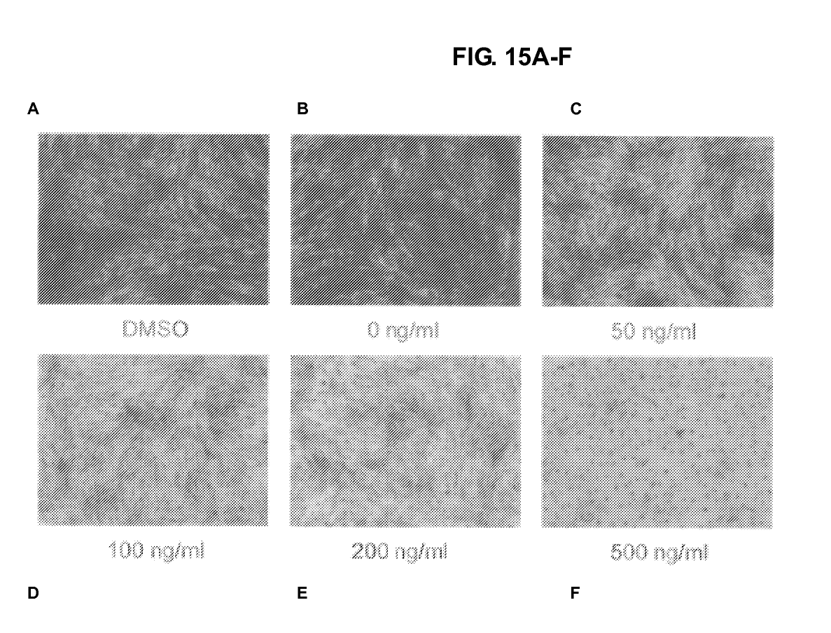

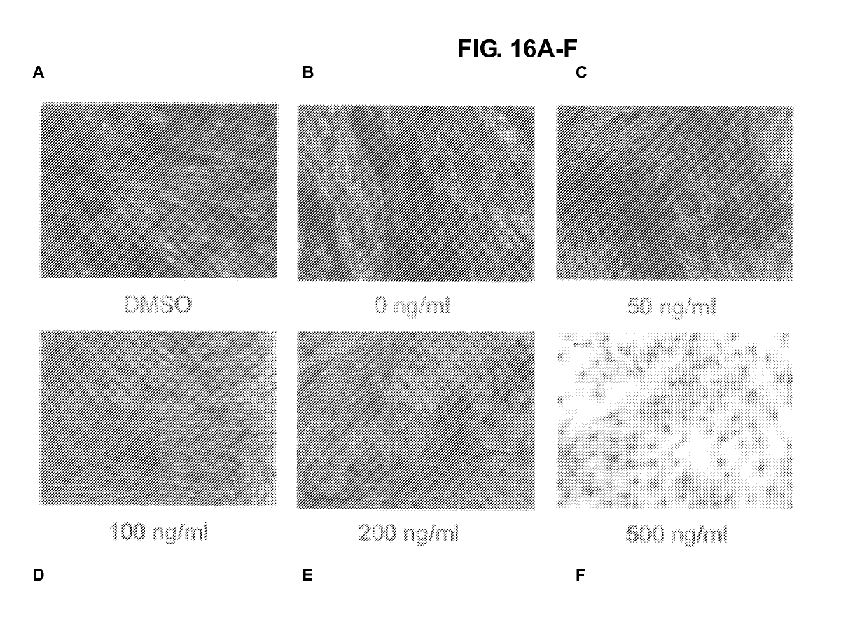

Furthermore, CCN2/CTGF-treated cells are neither osteogenic nor chondrogenic. Von Kossa staining was negative in CCN2/CTGF-treated MSCs (see e.g., FIG. 7B), just as MSCs without CCN2/CTGF treatment (see e.g., FIG. 7A). In contrast, MSCs subjected to osteogenic stimulation readily differentiated into osteogenic cells that elaborated minerals (see e.g., FIG. 7C). Safranin O staining was negative in CCN2/CTGF-treated MSCs (see e.g., FIG. 7E), just as MSCs without CCN2/CTGF treatment (see e.g., FIG. 7D). In contrast, MSCs subjected to chondrogenic stimulation readily differentiated into chondrogenic cells that were safranin O positive (see e.g., FIG. 7F). Quantitatively, MSCs under osteogenic stimulation elaborated significantly more calcium than the same subpopulation of cells with or without CCN2/CTGF treatment (see e.g., FIG. 7G) (n=5,*:p<0.05, **:p<0.01). In parallel, MSCs under chondrogenic stimulation produced significantly more glycosaminoglycans (GAG) than the same subpopulation of cells with or without CCN2/CTGF treatment (see e.g., FIG. 7H) (n=5, **:p<0.01).

### Example 15

### CCN2/CTGF-Derived Fibroblasts are aSMA-Negative Cells

Methods are according to Examples 1-14 unless otherwise specified.

Alpha smooth muscle actin (αSMA) is a pivotal hallmark of myofibroblasts that are activated from αSMA-negative fibroblasts among other cell types. Gain of αSMA by myofibroblasts is believed to have functional significance in dermal wound healing, cancer stroma and organ fibrosis. Given that αSMA was undetectable in CCN2/CTGF-stimulated MSCs (see e.g., FIG. 1G), this further analysis of αSMA expression was performed with a known stimulant for myofibroblast phenotype, TGFβ1.

Results showed that CCN2/CTGF-treated MSCs did not express αSMA (see e.g., FIG. 3A,C). Yet, 5 ng/mL TGFβ1 treatment of CCN2/CTGF-treated MSCs or MSC-derived fibroblastic cells readily expressed αSMA (see e.g., FIG. 3B,D). Flow cytometry confirmed the general absence of αSMA in MSCs with or without CCN2/CTGF treatment (see e.g., FIG. 3E,G, respectively).

Furthermore, 31.9% of TGFβ1-treated, MSC-derived fibroblastic cells gained αSMA phenotype (see e.g., FIG. 3H). Yet, only 1.8% of MSCs without prior CCN2/CTGF treatment gained αSMA phenotype (see e.g., FIG. 3F) after TGFβ1 stimulation. This shows that MSCs, without fibroblastic differentiation, may have some level of innate ability to resist the acquisition of αSMA phenotype, in comparison to CCN2/CTGF-treated MSCs.

In an established collagen gel contraction model, it was found that MSCs with sequential exposure to CCN2/CTGF and TGFβ1 yielded the most significant contraction (see e.g., FIG. 3I), in comparison with CCN2/CTGF stimulation alone (see e.g., FIG. 3J), or TGFβ1 stimulation alone (see e.g., FIG. 3K). MSCs without either TGFβ1 or CCN2/CTGF stimulation were least capable of contracting collagen gels (see e.g., FIG. 3L). Quantitative data confirmed that sequential stimulation of MSCs by CCN2/CTGF and TGFβ1 indeed yielded myofibroblast-like cells that were most capable of contracting collagen gel data (see e.g., FIG. 3M).

### Example 16

### CCN2/CTGF Favors Fibrogenesis Rather Than Ectopic Osteogenesis in Connective Healing

In this example, craniosynostosis was used as an in vivo model to test whether CCN2/CTGF, given its above-described in vitro efficacy on prompting fibrogenic fate of multipotent mesenchymal cells, is capable of defining the outcome of connective tissue healing.

Methods are according to Examples 1-15 unless otherwise specified.

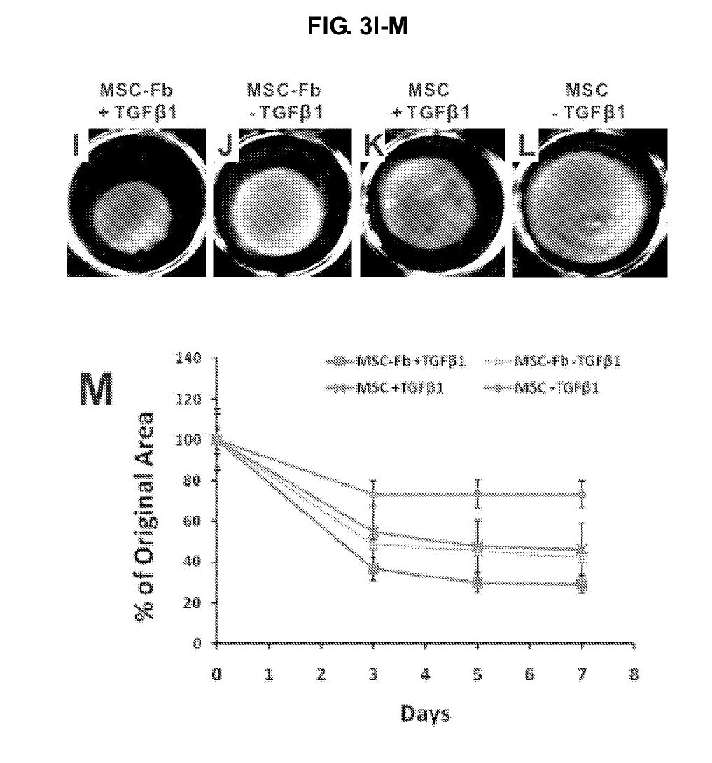

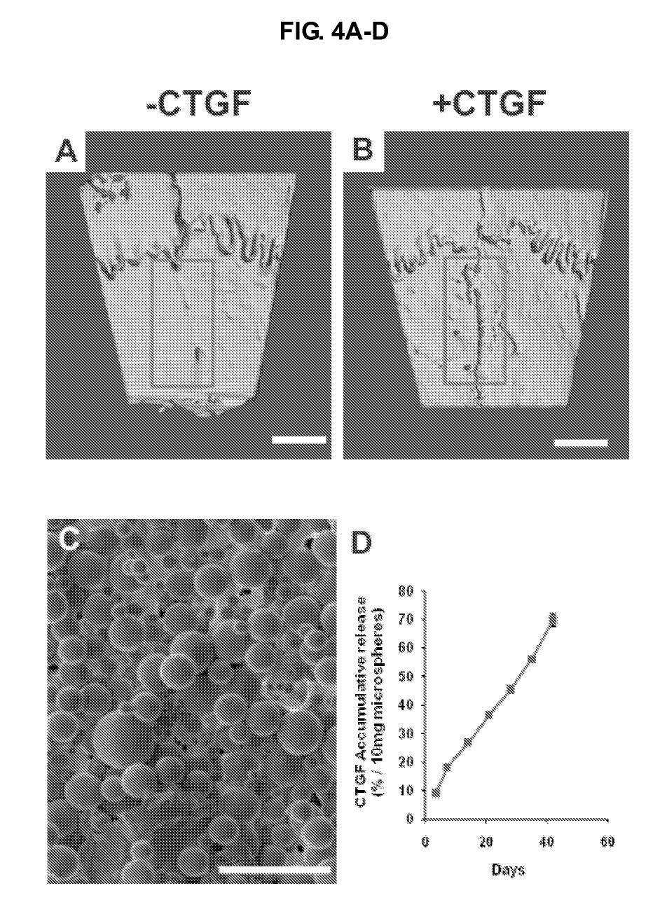

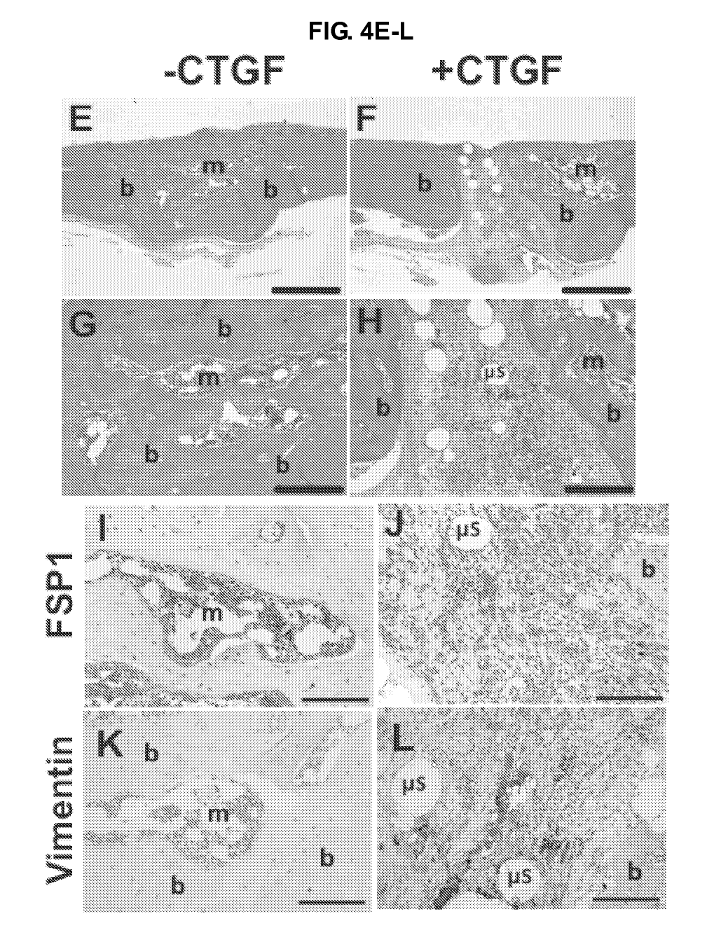

A control-release approach potentiated the bioactivity of CCN2/CTGF in vivo by microencapsulation (32), given rapid denature and diffusion of delivery of bioactive cues by injection (33). The in vitro release profile of microencapsulated CCN2/CTGF is shown in, for example, FIG. 4D.

Upon surgical removal of a synostosed calvarial suture in an established rat craniosynostosis model (34, 35), the outcome of tissue repair was re-synostosis (see e.g., FIG. 4A) with completely obliterated ectopic bone (see e.g., FIG. 4E,G), similar to re-synostosis in craniosynostosis patients (34). Strikingly, control-released CCN2/CTGF (see e.g., FIG. 4C,D) alone prompted fibrogenesis and restored the morphogenesis of an anatomic structure reminiscent of a native calvarial suture (see e.g., FIG. 4B; cf. (44)). CCN2/CTGF delivery by controlled release further restored microscopic characteristics of calvarial suture with mesenchymal- and fibroblast-like cells in the soft tissue interface between mineralized bone (see e.g., FIG. 4F,H). The presence of microspheres in bioengineered soft tissue interface (see e.g., μs in FIG. 4F,H) indicates that microencapsulated CCN2/CTGF was continuously released. Importantly, control-released CCN2/CTGF induced abundant FSP1 and vimentin expression in the restored calvarial suture (FIG. 4J,L), in comparison with the presence of FSP1 positive cells in the marrow of obliterated bone (FIG. 4I), and the general absence of vimentin without CCN2/CTGF delivery (FIG. 4I,K).

These findings demonstrate that controlled release of CCN2/CTGF may have implications in correcting ectopic mineralization in cardiac, orthopedic, vascular and other soft tissue defects.

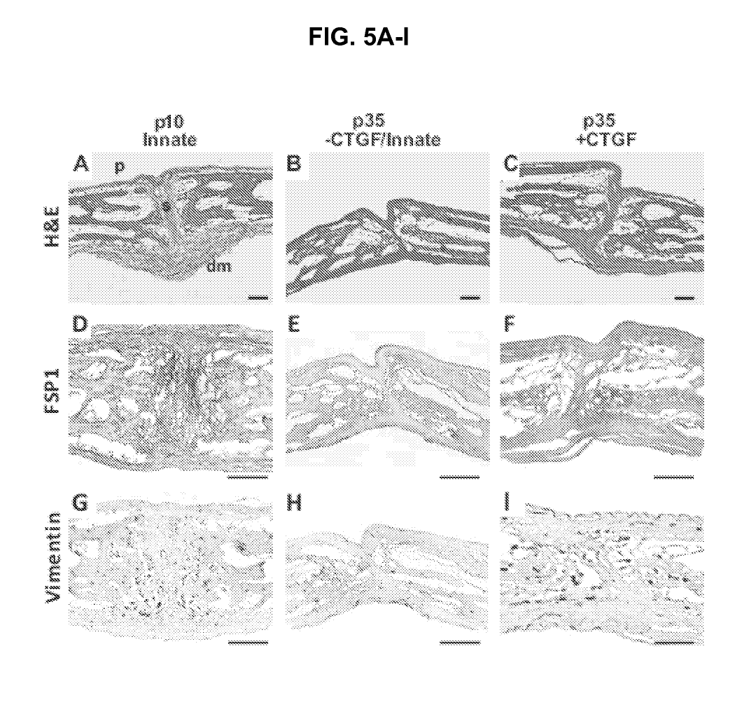

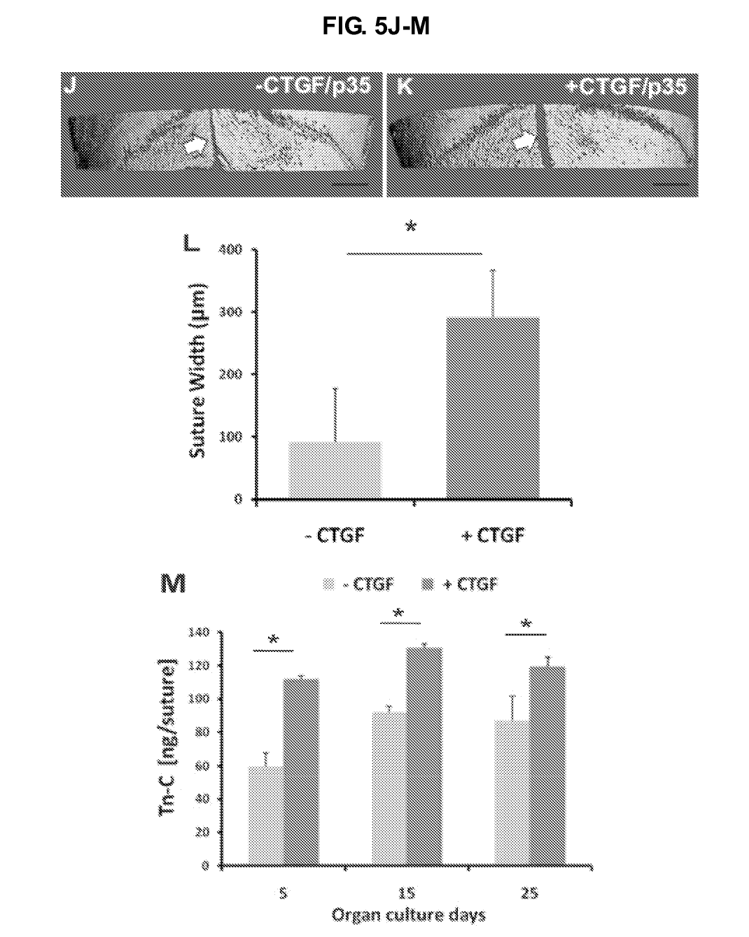

Ex vivo culture of the same-age calvaria (as for in vivo experiments above) using an established model (35) revealed substantially similar findings to the above-described in vivo craniosynostosis model. At postnatal day 10 (p10), a patent calvarial suture was characterized by the presence of mesenchymal/fibroblastic tissue between two bone formation fronts (see e.g., FIG. 5A), and the expression of FSP1 and vimentin (see e.g., FIG. 5D,G, respectively). Without CCN2/CTGF delivery in ex vivo culture, calvarial suture readily underwent synostosis by p35 (see e.g., FIG. 5B), along with diminished FSP1 and vimentin expression (see e.g., FIG. 5E,H, respectively). Contrastingly, 100 ng/mL CCN2/CTGF delivery rescued calvarial suture from undergoing synostosis, along with FSP1 and vimentin expression (see e.g., FIG. 5C,F,I, respectively). CCN2/CTGF-rescued calvarial suture showed patency, in contrast to virtual closure in CCCN2/CTGF-free sutures by pCT (see e.g., FIG. 5J,K), which is confirmed by quantitative analysis showing CCN2/CTGF delivery yielded significantly greater suture width than without CCN2/CTGF (see e.g., FIG. 5L). Tenacin C content was significantly greater in CCN2/CTGF rescued calvarial sutures than CCN2/CTGF-free sutures (see e.g., FIG. 5M), further showing that CCN2/CTGF prompted fibrogenesis.

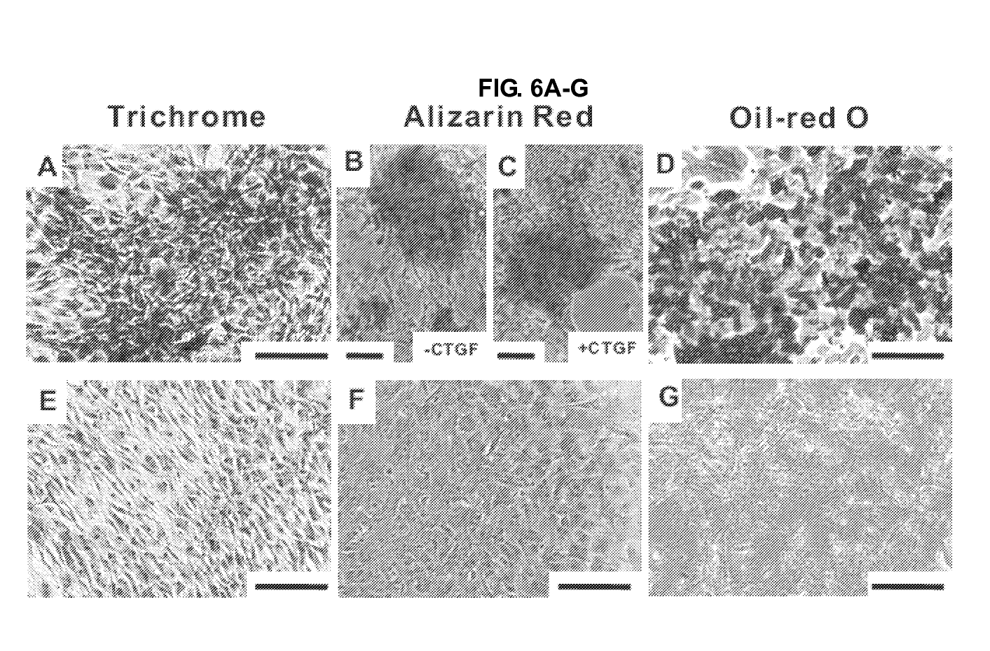

Furthermore, cells isolated from native, patent calvarial sutures by p7 readily differentiated into fibroblast-like cells that are highly Trichrome positive upon 100 ng/mL CCN2/CTGF stimulation (see e.g., FIG. 6A), demonstrating that multipotent mesenchymal cells in either appendicular bone marrow (as described above) or calvarium are capable of fibroblastic differentiation. In contrast, isolated calvarial suture cells without CCN2/CTGF treatment continued to assume MSC morphology and synthesized little collagen (see e.g., FIG. 6E). Cells isolated from p7 calvarial suture that was about to undergo synostosis within 20-30 days readily differentiated into osteoblasts under osteogenic stimulation with or without CCN2/CTGF (see e.g., FIG. 6B,C), in comparison to isolated cells without osteogenic stimulation (see e.g., FIG. 6F). Also, isolated calvarial suture cells underwent adipogenic differentiation under permissive conditions (see e.g., FIG. 6D), in comparison with isolated cells without adipogenic stimulation (see e.g., FIG. 6G).

These findings show that calvarial suture is constituted of multipotent mesenchymal cells that readily differentiate into fibroblastic cells and undergo fibrogenesis upon CCN2/CTGF stimulation, in addition to differentiation into other mesenchymal lineages.

### REFERENCES

1. H. Y. Chang et al., Proc Natl Acad Sci USA 99, 12877 (Oct. 1, 2002).
2. N. A. Bhowmick et al., Science 303, 848 (Feb. 6, 2004).
3. N. A. Bhowmick, E. G. Neilson, H. L. Moses, Nature 432, 332 (Nov. 18, 2004).
4. A. E. Karnoub et al., Nature 449, 557 (Oct. 4, 2007).
5. J. George, M. Tsutsumi, Gene therapy 14, 790 (May, 2007).
6. T. Kisseleva, D. A. Brenner, Exp Biol Med (Maywood) 233, 109 (February, 2008).
7. M. Ruiz-Ortega, J. Rodriguez-Vita, E. Sanchez-Lopez, G. Carvajal, J. Egido, Cardiovascular research 74, 196 (May 1, 2007).
8. R. F. Wynn et al., Blood 104, 2643 (Nov. 1, 2004).
9. I. A. Darby, T. D. Hewitson, International review of cytology 257, 143 (2007).
10. G. C. Gurtner, S. Werner, Y. Barrandon, M. T. Longaker, Nature 453, 314 (May 15, 2008).
11. A. Leask, Cell Signal 20, 1409 (August, 2008).
12. B. Hinz, J Invest Dermatol 127, 526 (March, 2007).
13. F. Van Eijk et al., Tissue Eng 10, 893 (May-June, 2004).
14. G. Vunjak-Novakovic, G. Altman, R. Horan, D. L. Kaplan, Annu Rev Biomed Eng 6, 131 (2004).
15. S.-Y. Woo et al., in Orthopaedic Basic Science J. A. Buckwalter, Einhon, T. A., Simon, S. R., Ed. (American Academy of Orthopaedic Surgeons, 2001).
16. K. Okita, T. Ichisaka, S. Yamanaka, Nature 448, 313 (Jul. 19, 2007).
17. J. Yu et al., Science 318, 1917 (Dec. 21, 2007).
18. D. Herzlinger, J Clin Invest 110, 305 (August, 2002).
19. R. Kalluri, E. G. Neilson, J Clin Invest 112, 1776 (December, 2003).
20. H. Robertson, J. A. Kirby, W. W. Yip, D. E. Jones, A. D. Burt, Hepatology 45, 977 (April, 2007).
21. J. L. Xia, C. Dai, G. K. Michalopoulos, Y. Liu, Am J Pathol 168, 1500 (May, 2006).
22. M. Zeisberg, R. Kalluri, J Mol Med 82, 175 (March, 2004).
23. M. Iwano et al., J Clin Invest 110, 341 (August, 2002).
24. M. Zeisberg, E. G. Neilson, J Clin Invest 119, 1429 (June, 2009).
25. Y. Bi et al., Nat Med 13, 1219 (October, 2007).
26. A. I. Caplan, J Orthop Res 9, 641 (September, 1991).
27. R. J. McAnulty, Int J Biochem Cell Biol 39, 666 (2007).
28. D. J. Prockop, Science 276, 71 (Apr. 4, 1997).
29. A. Bellini, S. Mattoli, Lab Invest 87, 858 (September, 2007).
30. A. Alhadlaq, J. J. Mao, Stem cells and development 13, 436 (August, 2004).
31. N. W. Marion, J. J. Mao, Methods in enzymology 420, 339 (2006).
32. E. K. Moioli, L. Hong, J. Guardado, P. A. Clark, J. J. Mao, Tissue engineering 12, 537 (March, 2006).
33. E. K. Moioli, P. A. Clark, X. Xin, S. Lal, J. J. Mao, Adv Drug Deliv Rev 59, 308 (May 30, 2007).
34. M. P. Mooney, A. M. Moursi, L. A. Opperman, M. I. Siegel, Expert opinion on biological therapy 4, 279 (March, 2004).
35. L. A. Opperman, R. W. Passarelli, E. P. Morgan, M. Reintjes, R. C. Ogle, J Bone Miner Res 10, 1978 (December, 1995).
36. E. G. Neilson, D. Plieth, C. Venkov, Trans Am Clin Climatol Assoc 114, 87 (2003).
37. E. K. Moioli et al., PLoS One 3, e3922 (2008).
38. I. L. Weissman, J. A. Shizuru, Blood 112, 3543 (Nov. 1, 2008).
39. J. J. Mao, Biol Cell 97, 289 (May, 2005).
40. E. M. Zeisberg et al., Nat Med 13, 952 (August, 2007).
41. X. Shi-Wen, A. Leask, D. Abraham, Cytokine Growth Factor Rev 19, 133 (April, 2008).
42. S. Friedrichsen et al., Cell Tissue Res 312, 175 (May, 2003).
43. S. Ivkovic et al., Development 130, 2779 (June, 2003).
44. E. K. Moioli, P. A. Clark, D. R. Sumner, J. J. Mao, Bone 42, 332 (February, 2008).
45. L. A. Opperman, A. M. Moursi, J. R. Sayne, A. M. Wintergerst, Anat Rec 267, 120 (Jun. 1, 2002).
46. Q. Luo et al., J Biol Chem 279, 55958 (Dec. 31, 2004).
47. O. A. Gressner, A. M. Gressner, Liver Int 28, 1065 (September, 2008).
48. B. Perbal, Lancet 363, 62 (Jan. 3, 2004).
49. S. Hankemeier et al., Tissue Eng 11, 41 (January-February, 2005).
50. J. E. Moreau et al., J Orthop Res 23, 164 (January, 2005).
51. A. Alhadlaq et al., Annals of biomedical engineering 32, 911 (July, 2004).
52. A. Alhadlaq, M. Tang, J. J. Mao, Tissue Eng 11, 556 (March-April, 2005).
53. M. S. Stosich et al., Tissue engineering 13, 2881 (December, 2007).
54. L. Lu, M. J. Yaszemski, A. G. Mikos, J Bone Joint Surg Am 83-A Suppl 1, S82 (2001).

SEQUENCE LISTING

SEQ ID NO: 1
Connective Tissue Growth Factor precursor [Homo sapiens]
ACCESSION NP_001892

| | |
|---|---|
| 1 | mtaasmgpvr vafvvllalc srpavgqncs gpcrcpdepa prcpagvslv ldgcgccrvc |
| 61 | akqlgelcte rdpcdphkgl fcdfgspanr kigvctakdg apcifggtvy rsgesfqssc |
| 121 | kyqctcldga vgcmplcsmd vrlpspdcpf prrvklpgkc ceewvcdepk dqtvvgpala |
| 181 | ayrledtfgp dptmirancl vqttewsacs ktcgmgistr vtndnascrl ekqsrlcmvr |
| 241 | pceadleeni kkgkkcirtp kiskpikfel sgctsmktyr akfcgvctdg rcctphrttt |
| 301 | lpvefkcpdg evmkknmmfi ktcachyncp gdndifesly yrkmygdma |

1-76. (canceled)

77. A pharmaceutical composition comprising:
CCN2/CTGF;
at least one of (i) an inhibitor of TGFβ, (ii) a P38 inhibitor, and (iii) a tyrosine kinase inhibitor;
a pharmaceutically acceptable carrier or excipient; and
optionally, a mesenchymal progenitor cell.

78. The composition of claim 77, wherein the mesenchymal progenitor cell is a αSMA− mesenchymal progenitor cell or a CD34− mesenchymal progenitor cell.

79. The composition of claim 77, wherein
the CCN2/CTGF comprises a CCN2/CTGF polypeptide;
the CCN2/CTGF comprises a polynucleotide encoding a CCN2/CTGF polypeptide;
the composition comprises a polynucleotide encoding a CCN2/CTGF polypeptide operably linked to a vector suitable for expression of the CCN2/CTGF polypeptide in a wound tissue environment;
the CCN2/CTGF comprises human CCN2/CTGF or recombinant human CCN2/CTGF;
the CCN2/CTGF comprises a CCN2/CTGF corresponding to Accession No. NP_{—}001892; or
the CCN2/CTGF comprises a polypeptide having a sequence of SEQ ID NO: 1, or at least about 95% identity thereto and CCN2/CTGF activity.

80. The composition of claim 77, wherein the composition comprises an inhibitor of TGFβ and at least one of the following features is satisfied:
the inhibitor of TGFβ reduces formation of myofibroblasts from fibroblasts or inhibits fibrosis;
the inhibitor of TGFβ substantially reduces formation of myofibroblasts from fibroblasts or inhibits fibrosis;
the inhibitor of TGFβ is an inhibitor of TGFβ1; and
the inhibitor of TGFβ is selected from the group consisting of ANG-1122, AP-11014, metelimumab, fresolimumab, mannose-6-phosphate, Pharmaprojects No. 6614, NAFB001, NAFB002, TGF-β1 antibody, LY-2157299, Fetuin, TGF-β antagonists, 1D11, anti-TGFβ MAb-1, SB-431542, activin-like kinase 5 inhibitor, anti-TGF-β antibodies, antisense oligonucleotide, TGF-β receptor, decorin, SX-007, TGF-β receptor inhibitors, TGF-β vaccine, ADMP-1, TGF-β antibodies, mannose-6-phosphonate, cancer gene therapy, TGF-Beta Shield, IN-1130, LF-984, TGF-β inhibitors, and SB-431542.

81. The composition of claim 77, wherein the composition comprises a P38 inhibitor selected from the group consisting of Tocriset, SD282, SB239063, SB203580, SB220025, SKF86002, PD169316, SB202190, SC68376, VX702, VX745, R130823, AMG548, BIRB796, SCIO469, SCIO323, FR167653, MW012069ASRM, SD169, RWJ67657, and ARRY797.

82. The composition of claim 77, wherein the composition comprises a tyrosine kinase inhibitor selected from the group consisting of K252a, Axitinib, Bosutinib, Cediranib, Dasatinib, Erlotinib, Gefitinib, Imatinib, Lapatinib, Lestaurtinib, Nilotinib, Semaxanib, Sunitinib, Toceranib, Vandetanib, Vatalanib, ZD 1839, CI-1033, OSI-774, GW 2016, EKB-569, IMC-C225, MDX-447, PKI 116, ABX-EGF, AG-82, AG-18, AG-490, AG-17, AG-213, AG-494, AG-825, AG-879, AG-1112, AG-1296, AG-1478, AG-126, RG-13022, RG-14620, and AG-555.

83. The composition of claim 77, further comprising an antibiotic or an immunosuppressive agent; and, optionally (i) the antibiotic is selected from the group consisting of amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin; or (ii) the immunosuppressive agent selected from the group consisting of a steroid, cyclosporine, cyclosporine analog, cyclophosphamide, methylprednisone, prednisone, azathioprine, FK-506, 15-deoxyspergualin, prednisolone, methotrexate, thalidomide, methoxsalen, rapamycin, leflunomide, mizoribine, brequinar, deoxyspergualin, azaspirane, muromonab-CD3, Sandimmune, Neoral, Sangdya, Prograf, Cellcept, azathioprine, glucocorticosteroids, adrenocortical steroid, Deltasone, Hydeltrasol, Folex, methotrexate, methoxsalen, and sirolimus.

84. The composition of claim 77, wherein the composition is:
formulated for parenteral, pulmonary, oral, topical, intradermal, intramuscular, intraperitoneal, intravenous, subcutaneous, intranasal, epidural, ophthalmic, buccal, or rectal administration;
formulated for topical administration; or
formulated for topical administration directly to a soft tissue wound site.

85. A system for healing a wound at a tissue site, comprising:
a medical device; and
a composition of claim 77;
wherein
the composition is adapted to be released from the medical device when in contact with a tissue site, and
optionally, the medical device is selected from the group consisting of a drape, bandage, dressing, tape, adhesive layer, splint, blood stop powder, steri strip, cyanoacrylate glue, staple, suture, and combinations thereof.

86. A method of treating a subject comprising:
administering to a tissue wound site in a subject in need thereof
(i) a pharmaceutical composition of claim 77;
(ii) a system of claim 85; or
(iii) a first composition comprising CCN2/CTGF and a second composition comprising at least one of
(a) an inhibitor of TGFβ,
(b) a P38 inhibitor, and
(c) a tyrosine kinase inhibitor,
wherein the second composition inhibits fibrosis.

87. The method of claim 86, wherein administering the pharmaceutical composition comprises contacting the pharmaceutical composition and a mesenchymal progenitor cell to stimulate fibroblast differentiation, optionally, to αSMA− fibroblasts or FSP1+, vimentin+, Coll1+ and αSMA− fibroblasts.

88. The method of claim 86, wherein the first composition and the second composition are administered consecutively or simultaneously.

89. The method of claim 86, wherein one or more of the following features are satisfied:
the inhibitor of TGFβ reduces formation of myofibroblasts from fibroblasts or inhibits fibrosis;
the inhibitor of TGFβ is present in an amount effective to substantially reduce formation of myofibroblasts from fibroblasts;
the inhibitor of TGFβ is an inhibitor of TGFβ1;
the inhibitor of TGFβ is selected from the group consisting of ANG-1122, AP-11014, metelimumab, fresolimumab, mannose-6-phosphate, Pharmaprojects No. 6614, NAFB001, NAFB002, TGF-β 1 antibody, LY-2157299, Fetuin, TGF-β antagonists, 1D11, anti-TGFβ MAb-1, SB-431542, activin-like kinase 5 inhibitor, anti-TGF-β antibodies, antisense oligonucleotide, TGF-β receptor, decorin, SX-007, TGF-β receptor inhibitors, TGF-β vaccine, ADMP-1, TGF-β antibodies, mannose-6-phosphonate, cancer gene therapy, TGF-Beta Shield, IN-1130, LF-984, TGF-β inhibitors, and SB-431542.
the P38 inhibitor is selected from the group consisting of Tocriset, SD282, SB239063, SB203580, SB220025, SKF86002, PD169316, SB202190, SC68376, VX702, VX745, R130823, AMG548, BIRB796, SCIO469, SCIO323, FR167653, MW012069ASRM, SD169, RWJ67657, and ARRY797; and
the tyrosine kinase inhibitor is selected from the group consisting of K252a, Axitinib, Bosutinib, Cediranib, Dasatinib, Erlotinib, Gefitinib, Imatinib, Lapatinib, Lestaurtinib, Nilotinib, Semaxanib, Sunitinib, Toceranib, Vandetanib, Vatalanib, ZD 1839, CI-1033, OSI-774, GW 2016, EKB-569, IMC-C225, MDX-447, PKI 116, ABX-EGF, AG-82, AG-18, AG-490, AG-17, AG-213, AG-494, AG-825, AG-879, AG-1112, AG-1296, AG-1478, AG-126, RG-13022, RG-14620, and AG-555.

90. The method of claim 86, wherein the tissue wound site comprises one or more of:
a soft tissue wound;
a chronic soft tissue wound or an acute soft tissue wound;
a dermal wound, a ligament wound, a tendon wound, or a combination thereof;
an open tissue wound;
an incision wound, a laceration wound, an abrasion wound, a puncture wound, a penetration wound, or a gunshot wound; and
a split laceration, over stretching, grinding compression, cut laceration, or tearing.

91. The method of claim 86 wherein administration comprises:
parenteral, pulmonary, oral, topical, intradermal, intramuscular, intraperitoneal, intravenous, subcutaneous, intranasal, epidural, ophthalmic, buccal, or rectal administration;
topical administration directly to the tissue wound site; or
administration via carrier delivery system, the carrier delivery system comprising polymeric microspheres encapsulating the composition.

92. The method of claim 91, wherein the composition is encapsulated in polymeric microspheres at a ratio of:
about 100 mg to about 500 mg polymer to about 1 μg to about 100 μg of CCN2/CTGF; or
about 250 mg polymer to about 10 μg of CCN2/CTGF.

93. The method of claim 92, wherein administering the composition comprises introducing about 1 mg to about 50 mg of CTGF-encapsulated microspheres to a tissue wound.

94. The method of claim 86, wherein administration results in at least one of (i) enhancement of fibroblast differentiation, (ii) enhancement of fibrogenesis, (iii) inhibition of myofibroblast differentiation, and (iv) inhibition of fibrosis.

95. A method of forming an αSMA− fibroblast comprising:
contacting a αSMA−, CD34− mesenchymal progenitor cell and CCN2/CTGF, wherein the CCN2/CTGF-stimulated mesenchymal stem cell differentiates into a αSMA−, FSP1+, vimentin-F, Coll1+ fibroblast cell.
